(19) United States
(12) Patent Application Publication
(10) Pub. No.: US 2026/0283613 A1
(43) Pub. Date: Sep. 24, 2026

(54) SURGICAL STAPLING INSTRUMENTS AND CONTROL SYSTEMS FOR SUCH INSTRUMENTS

(71) Applicant: INTUITIVE SURGICAL OPERATIONS, INC., Sunnyvale, CA (US)

(72) Inventor: Matthew Wixey, Rochester, MI (US)

(73) Assignee: INTUITIVE SURGICAL OPERATIONS, INC., Sunnyvale, CA (US)

(21) Appl. No.: 19/474,225

(22) PCT Filed: Apr. 10, 2024
(86) PCT No.: PCT/US2024/023816

§ 371 Date: Oct. 10, 2025

Related U.S. Application Data
(60) Provisional application No. 63/458,510, filed on Apr. 11, 2023.

(51) Int. Cl. A61B 17/072

(52) U.S. Cl. CPC A61B 17/07207; A61B 2017/07257; A61B 2017/07271

(57) ABSTRACT

Surgical stapling instruments and control systems for use with those instruments are provided herein. A surgical stapling instrument comprises an elongate shaft and an end effector with an anvil on a first jaw and a staple cartridge coupled to a second jaw. The instrument further includes a first coupler for adjusting a distance between the distal ends of the anvil and the cartridge and a second coupler for adjusting a distance between the proximal ends of the anvil and the cartridge. This allows the user to independently adjust these distances or gaps to account for variations in tissue thickness across the length of the jaws. The instruments and control systems are particularly useful for dissecting and stapling a relatively long length of tissue, such as the stomach or the lung in sleeve gastrectomy and lung resection procedures.

### CROSS-REFERENCE TO RELATED APPLICATIONS

This application claims the benefit of U.S. Provisional Application Ser. No. 63/458,510, filed Apr. 11, 2023, the complete disclosure of which is incorporated herein by reference for all purposes.

### BACKGROUND

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries use these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

One example of a laparoscopic procedure is sleeve gastrectomy. Sleeve gastrectomy is a surgical weight-loss procedure in which the stomach is reduced to about 15% of its original size, by surgical removal of a large portion of the stomach along the greater curvature. The result is a sleeve or tube like structure. The procedure permanently reduces the size of the stomach, although there could be some dilation of the stomach later on in life.

The sleeve gastrectomy procedure involves a longitudinal resection of the stomach starting from the antrum at the point about 5-6 cm from the pylorus and finishing at the fundus close to the cardia. The remaining gastric sleeve is calibrated with a bougie. Most surgeons prefer to use a bougie between 36 and 40 Fr with the procedure and the ideal approximate remaining size of the stomach after the procedure is about 150 mL.

Although sleeve gastrectomy procedures have become more common, differences in operative techniques such as oversewing, bougie size, and distance of the staple line from the pylorus may lead to less than optimal outcomes. Ideal tubular sleeve anatomy is achieved in less than 40% of radiologically studied sleeves resulting in variable outcomes for the patients, including reduced weight loss efficiency and gastroesophageal reflux. As a result, recent efforts have been made towards standardization of the procedure in order to improve patient outcomes, decrease operative time, and reduce costs.

One such effort towards increasing the consistency of sleeve gastrectomy procedures are single-fire bariatric stapler devices that have been developed to dissect and staple the entire length of the gastric greater curvature with one staple load. These single-fire stapler devices have much longer jaws than traditional staplers to allow the surgeon to essentially dissect and staple the entire gastric greater curvature by clamping the jaws from one end of the greater curvature to the other. The proposed benefits of these new stapler devices include decreased operative time, removal of junctions in the staple line, and elimination of angulation between staple loads. Without multiple crossed staple lines, these single-fire staplers may eliminate the surgeons' search for and removal of the migratory “crotch staple” which has been identified as a risk of leak.

While the new single-fire stapler devices have shown promise, they suffer from a number of drawbacks. One such drawback is that these staplers do not account for variations in tissue thickness across the length of the jaws. For example, if the thickness of the distal portion of the stomach tissue (relative to the instrument) is substantially different from the proximal portion of the stomach tissue (or vice versa), the stapler may not effectively clamp down on the entire length of tissue. Alternatively, the stapler may be urged by the stomach tissue to clamp down in a direction that is offset from, or transverse to, the desired direction, resulting in an ineffective or suboptimal dissection and stapling line.

Another drawback with existing single-fire stapler devices is that the jaws of these devices are generally fixed with respect to the shaft of the instrument. They do not possess any degrees of rotational movement relative to the shaft that would mimic the natural action of a surgeon's wrist. This inability to rotate the end effector relative to the shaft makes it challenging for a surgeon to position the jaws in the optimal location for performing the dissection/stapling function, particularly in a laparoscopic procedure wherein the instrument has been inserted through a small entry point into the abdominal cavity.

Accordingly, while the new bariatric stapler devices have improved certain procedures that involve the dissection of relatively long tissue lengths, such as stomach tissue in sleeve gastrectomy procedures, still further improvements would be desirable to overcome the drawbacks with these instruments. The systems and devices described herein address these and other needs.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

Surgical stapling instruments and control systems for use with those instruments are provided herein. The instruments and control systems are particularly useful for dissecting and/or stapling a relatively long length of tissue, such as the stomach or the lung in sleeve gastrectomy and or lung resection procedures.

In one aspect, a surgical stapling instrument comprises an elongate shaft having distal and proximal ends and an end effector coupled to the distal end of the shaft. The end effector comprises an anvil on a first jaw having a surface positionable on a first side of an anatomical structure and a cartridge coupled to a second jaw and configured to house a plurality of staples and having a surface positionable on a second side of the anatomical structure. The instrument further includes a first coupler for movably coupling the distal end of the anvil to the distal end of the cartridge and a second coupler for movably coupling the proximal end of the anvil to the proximal end of the cartridge.

In embodiments, the first and second couplers are configured to adjust a distance or gap between the proximal and distal ends of the anvil and the staple cartridge. The first and second couplers may be independent from each other. This allows the user to independently adjust the distances or gaps between the proximal and distal ends of the anvil and the staple cartridge to, for example, account for variations in tissue thickness across the length of the jaws.

In certain embodiments, the instrument further includes a drive member configured to translate through the end effector to drive staples from the cartridge into deforming contact with the anvil. The drive member may also include a cutting element that passes through the jaws to dissect tissue. Simultaneously with the dissection of tissue, staples are driven into the tissue on either side of the line of dissection.

In embodiments, the first coupler limits the range of distance between the distal ends of the anvil and the staple cartridge between a minimum distance and a maximum distance. The second coupler may also limit the range of distance between the proximal ends of the anvil and the staple cartridge between a minimum distance and a maximum distance. These ranges may be set, for example, based on the maximum and minimum distances that allow for adequate staple formation through a particular type of tissue once the staples have been driven against the anvil by the drive member.

The first and second couplers may be actuated and controlled manually through one or more user controls at the proximal end of the instrument, e.g., on an instrument handle or a user interface in, for example, a robotic control system. Alternatively, the first and second couplers may be actuated and controlled automatically through a control system, such as a robotic control system.

In certain embodiments, the couplers may be actuated and controlled manually by the user within a certain range that is monitored and controlled by a control system. This range may, for example, be based on a range of torques and/or forces applied to the components of the couplers and/or the jaws as the jaws are adjusted relative to each other. The ranges of torque or force may, for example, be based on forces applied by tissue positioned between the jaws (i.e., as the jaws are moved towards each other against the tissue). This allows the system to ensure that the distal and proximal ends of the jaws have a suitable gap therebetween for providing adequate tissue loads for good anastomosis when the drive member is translated through the end effector (i.e., “fired”) to dissect tissue and apply staples to either side of the line of dissection.

In one embodiment, the first or proximal coupler comprises a pulley and a band having a planar first surface in contact with the pulley and a planar second surface opposite the first surface. The band has a distal end coupled to the proximal end of the anvil and a second portion of the band is configured for movement away from this distal end to adjust the distance between the proximal ends of the anvil and the cartridge. In embodiments, movement of the band also causes the proximal end of the anvil to pivot relative to the proximal end of the cartridge from an open position to a substantially closed position.

The second or distal coupler may also comprise a pulley and a band having a planar first surface in contact with the pulley and a planar second surface opposite the first surface. The band has a distal end coupled to the distal end of the anvil and a second portion of the band is configured for movement away from this distal end to adjust the distance between the distal ends of the anvil and the cartridge. In certain embodiments, the instrument further comprises a slot in a distal portion of the cartridge and/or the lower jaw, and a pin slidable within the slot. The pin is coupled to the anvil to allow the distal end of the anvil to move towards and away from the distal end of the cartridge. In certain embodiments, the slot extends in a direction substantially perpendicular to the longitudinal axis of the end effector. This allows the jaws to pivot about their distal ends to open and close the proximal ends, while independently adjusting the gap between the distal and/or proximal ends of the anvil and the cartridge.

In certain embodiments, the instrument further comprises a first spring biasing the distal ends of the anvil and the cartridge away from each other and/or a second spring biasing the proximal ends of the anvil and the cartridge away from each other. In this manner, the distal end and proximal ends of the anvil and cartridge are biased into positions wherein the gap therebetween is maximized (i.e., fully open positions). Tension may be applied to one or both of the bands by, for example, a proximal drive element to pull the bands proximally and reduce the gap or distance between either of the proximal or distal ends of the jaws.

In another embodiment, the proximal coupler comprises a drive element having an angled slot and a slot pin configured to advance through the angled slot to adjust a distance between the proximal and distal ends of the anvil and the cartridge. The drive element may, for example, be located within the staple cartridge, one of the jaws of the end effector, or the shaft of the instrument. The slot pin may be coupled to the movable jaw or anvil such that longitudinal movement of the drive element causes the movable jaw and/or anvil to open and close relative to the other jaw or staple cartridge. In certain embodiments, distal movement of the drive element causes the slot pin to advance proximally through the angled slot to close the proximal ends of the jaws.

The surgical instrument may further include one or more drive element(s) configured to advance (i.e. push and/or pull) the bands and to translate the drive member distally through the end effector. The drive element(s) may include one or more control devices of a robotic surgical system.

In another aspect, a surgical stapling instrument comprises an elongate shaft having distal and proximal ends and an end effector coupled to the distal end of the shaft. The end effector comprises an anvil on a first jaw having a surface positionable on a first side of an anatomical structure and a cartridge coupled to a second jaw, configured to house a plurality of staples and having a surface positionable on a second side of the anatomical structure. The surgical stapling instrument further comprises a coupler that movably couples the proximal end of the anvil to the proximal end of the cartridge and an articulation element for rotating the end effector relative to the shaft.

In certain embodiments, the articulation element is configured to rotate the end effector about the longitudinal axis of the shaft (i.e., a “roll” axis). In other embodiments, the end effector may be rotated about an axis substantially perpendicular to the longitudinal axis of the shaft, i.e., either a “yaw” or a “pitch” axis relative to the shaft. Providing at least one degree of rotational movement relative to the shaft enables the end effector to correspond with at least a portion of the natural action of a surgeon's wrist, thereby facilitating placement of the jaws in the optimal location for performing the dissection/stapling function, particularly in a laparoscopic procedure wherein the instrument has been inserted through a small entry point into the abdominal cavity.

In certain embodiments, the instrument comprises a second articulation element for rotating the end effector relative to the shaft about a second axis perpendicular to the first axis and perpendicular to the longitudinal axis. In this manner, the end effector may be rotated about both a yaw and a pitch axis and/or about a roll axis and either a yaw or a pitch axis. The instrument may comprise a third articulation element for rotating the end effector relative to the shaft about the longitudinal axis (i.e., roll, pitch and yaw).

In embodiments, the articulation element(s) comprise at least one band extending through the end effector and having a planar first surface and a planar second surface opposite the first surface. The articulation element may also extend into a wrist member that pivotally couples the shaft to the end effector. The band is configured to move laterally relative to the shaft within the wrist member when tension or compression is applied to the band from a proximal drive element, thereby rotating the end effector relative to the shaft.

In embodiments, the articulation element(s) comprise a plurality of bands that may, for example, be laminated to each other. The bands are configured to be twisted relative to each other within the wrist member, thereby causing the end effector to rotate relative to the shaft around an axis perpendicular to the longitudinal axis.

In embodiments, the instrument comprises a second coupler that movably couples the distal end of the anvil to the distal end of the cartridge. The first coupler may be independent of the second coupler such that the distances or gaps between the distal and proximal ends of the jaws may be independently adjusted.

In another aspect, systems and methods are provided for controlling a surgical stapling instrument having an elongate shaft and an end effector coupled to the distal end of the shaft, the end effector having an anvil and a cartridge having proximal and distal ends movably coupled to each other. The system comprises a first controller configured to detect a first torque and/or force applied to the proximal ends of the anvil and the cartridge and to maintain a distance between said proximal ends such that the first torque or force remains within a first range and a second controller configured to detect a second torque and/or force applied to the distal ends of the anvil and the cartridge and to maintain a distance between said distal ends such that the second torque or force remains within a second range.

The first and second controllers may be separate from each other, or part of the same controller. In some embodiments, the controllers are part of a robotic control system coupled to the surgical instrument.

In embodiments, the first and second torques are associated with one or more forces applied to the anvil and the cartridge by tissue disposed therebetween. The controller(s) may be configured to maintain the distances of the proximal and distal ends of the jaws within ranges based on these forces. In one such embodiment, for example, the ranges may be based on a minimum distance representing a threshold force applied by the tissue to the jaws such that the jaws do not compress the tissue too much to effectively apply staples thereto. The ranges may also be based on a maximum distance representing a threshold and/or zero amount of force applied by the tissue to the jaws such that the jaws are not separated so far from each other to be unable to effectively apply staples to the tissue.

In embodiments, the stapling instrument further comprises a drive member configured to advance in a longitudinal direction relative to the cartridge and the anvil to drive staples from the cartridge into deforming contact with the anvil. The system further comprising a third controller configured to inhibit or prevent the advancement of the drive member when either of the first or second torques are outside of the first and second ranges, respectively. The third controller may be independent of the first and/or second controllers, or the controllers may cooperate with each other as part of a single control system.

In certain embodiments, the system further includes a proximal coupler configured to adjust the distance between the proximal ends of the anvil and the cartridge. The first controller is coupled to the proximal coupler and configured to limit a range of motion of one or more of the components of the proximal coupler based on the first torque relative to the first range.

The system may further comprise a distal coupler for adjusting the distance between the distal ends of the anvil and the cartridge. The second controller is coupled to the distal coupler and configured to limit a range of motion of one or more of the components of the distal coupler based on the second torque relative to the second range.

In embodiments, the proximal and distal couplers each comprise a flexible member, such as a cable or band, having a distal end coupled to the proximal end of the anvil and a proximal end coupled to the first controller. The system further comprises a drive member for withdrawing at least a portion of the flexible member through the shaft to move the proximal or distal ends of the anvil towards the proximal or distal ends of the cartridge. The first and second controllers may be configured to detect the torque and/or force applied to the flexible member and/or to the jaws as the flexible member is withdrawn.

In another aspect, a method for controlling a surgical stapling instrument is provided. The method comprises detecting a first torque applied to the proximal ends of first and second jaws of an end effector of the surgical stapling instrument and detecting a second torque applied to the distal ends of the first and second jaws. The method further comprises releasing a drive member for advancement through the end effector when the first or second torques are within first and second ranges, respectively.

In embodiments, the method further comprises adjusting a distance or gap between the proximal ends of the anvil and the cartridge and detecting the first torque as this gap is adjusted. In one such embodiment, a flexible member is advanced relative to the end effector to adjust the gap between the proximal ends of the anvil and the cartridge and further movement of the flexible member is restricted or prevented when the first torque reaches a threshold level.

In embodiments, the method further comprises adjusting a distance or gap between the distal ends of the anvil and the cartridge and detecting the second torque as this gap is adjusted. In one such embodiment, a second flexible member is advanced relative to the end effector to adjust the distance between the distal ends of the anvil and the cartridge and further movement of the flexible member is restricted or prevented when the second torque reaches a threshold level.

The method may further comprise inhibiting or preventing the drive member from advancing through the end effector when either of the first or second torques is below a threshold minimum or above a threshold maximum.

In another aspect, a surgical stapling system comprises an instrument having an elongate shaft, an end effector coupled to the distal end of the shaft and a drive member configured to advance through the end effector. The end effector has an anvil on a first jaw and a staple cartridge coupled to a second jaw. The anvil and the staple cartridge have proximal and distal ends movably coupled to each other. The system further comprises a first coupler for adjusting a distance between the distal end of the anvil and the distal end of the cartridge, a second coupler for adjusting a distance between the proximal end of the anvil and the proximal end of the cartridge and a third actuator for advancing the drive member in a longitudinal direction relative to the anvil and the cartridge.

In embodiments, the system further comprises an articulation mechanism for rotating the end effector about a longitudinal axis of the end effector. This allows the surgeon to rotate the end effector without moving the shaft, which provides more flexibility for positioning the jaws in an optimal location for stapling tissue therebetween, particularly in an endoscopic procedure wherein the shaft extends through an entry point in the patient's skin to a cavity within the patient's body.

In embodiments, the system further comprises a second articulation mechanism for rotating the end effector relative to the shaft about an axis perpendicular to the longitudinal axis. In certain embodiments, the second articulation mechanism is configured to rotate the end effector about a yaw axis of the shaft. In other embodiments, the second articulation mechanism is configured to rotate the end effector about a pitch axis of the shaft. In other embodiments, the second articulation mechanism rotates the shaft about both the pitch and yaw axes. In an alternative embodiment, the system may comprise a third articulation mechanism that allows for independent rotation about each of the yaw and pitch axes.

In embodiments, the first and second couplers each comprise a band having a planar first surface and a planar second surface opposite the first surface and a gear disposed at a proximal end portion of the shaft, wherein the gear is coupled to a rotating disc and configured to translate rotation of the disc into linear movement of the band. In an exemplary embodiment, the system further comprises a controller for rotating the discs. The controller may be part of a robotic control system that allows for remote control of the instrument.

In embodiments, the first and second articulation mechanisms each comprise one or more bands that extend through the end effector and into a wrist element pivotally coupling the end effector to the shaft. The bands may be, for example, laminated with each other to form a composite band with additional rigidity. In an exemplary embodiment, the bands are coupled to a proximal drive element and configured to twist within the wrist to allow for rotational movement of end effector relative to the shaft. The proximal drive element may be part of a robotic control system that allows for remote control of the instrument.

### BRIEF DESCRIPTION OF THE DRAWINGS

The above and other aspects, features, and advantages of the present surgical instruments having a locking mechanism will become more apparent in light of the following detailed description when taken in conjunction with the accompanying drawings in which:

FIG. 1 is a perspective view of a representative surgical instrument having an end effector mounted to an elongated shaft, and an actuation mechanism;

FIG. 1A is a perspective view of representative surgical instrument with a robotically controlled backend mechanism;

FIG. 2 is a perspective view of the distal end portion of a representative surgical instrument with the jaws in the open position;

FIG. 3 is an exploded view of a cartridge configured for use with the surgical instrument of FIG. 1;

FIG. 4 is a cross-sectional view of the jaws, illustrating proximal and distal couplers;

FIG. 5 is an enlarged view of the distal end portion of the jaws;

FIG. 6 is a cross-sectional view of an alternative embodiment of a proximal coupler for the jaws of the surgical instrument;

FIGS. 7A and 7B are cross-sectional view of another embodiment of a proximal coupler for the jaws of the surgical instrument;

FIG. 8 is a cross-sectional view of another embodiment of a distal coupler for the jaws of the surgical instrument;

FIG. 9. is a cross-sectional view of another embodiment of a proximal coupler for the jaws of the surgical instrument;

FIG. 10A is a perspective view of a representative drive member of the surgical instrument of FIG. 1;

FIG. 10B is an alternative embodiment of a drive member;

FIG. 11 is a cross-sectional side of a representative clevis for the drive member of FIG. 10B;

FIG. 12A is a cross-sectional perspective view of an actuation mechanism for the drive member of FIG. 10B;

FIG. 12B is a cross-sectional side view of the actuation mechanism for the drive member of FIG. 10B;

FIG. 13 is a cross-sectional view of a shaft of the surgical instrument, illustrating a plurality of articulation bands;

FIG. 14A is a cross-sectional view of the bands of FIG. 13 rotated within a wrist of the surgical instrument;

FIG. 14B is another cross-sectional view of the bands of FIG. 13 articulating the end effector of the surgical instrument;

FIG. 15 is an enlarged view of a representative robotic backend mechanism for the surgical instrument;

FIG. 16 illustrates a drive mechanism for an articulation band of the robotic backend mechanism;

FIG. 17 is a schematic view of a control system for a surgical instrument;

FIG. 18 depicts the anatomy of a stomach;

FIG. 19 illustrates an end effector of a surgical instrument as described herein positioned on the stomach;

FIG. 20 illustrates the end effector of FIG. 19 following resection of the stomach;

FIG. 21 illustrates a top view of an operating room employing a robotic surgical system; and

FIG. 22 illustrates a simplified side view of a robotic arm assembly.

### DETAILED DESCRIPTION

Particular embodiments of the present surgical instruments are described hereinbelow with reference to the accompanying drawings; however, it is to be understood that the disclosed embodiments are merely exemplary of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Well-known functions or constructions are not described in detail to avoid obscuring the present disclosure in any unnecessary detail.

While the following description is presented with respect to a bariatric surgical stapler device and control system for use in stapling and dissecting stomach tissue in a sleeve gastrectomy procedure, it should be understood that features of the presently described surgical instruments and/or control systems may be readily adapted for use in any type of surgical clamping, cutting, stapling, ligating, dissecting, clipping, cauterizing, suturing and/or sealing instrument, whether or not the surgical instrument applies a fastener. For example, the presently described stapling devices and control systems may be adapted for use in other procedures involving anatomical structures, such as organs other than the stomach or soft tissue, e.g., lung tissue, liver tissue and the like.

In another example, some of the presently described features may be used in an electrosurgical instrument wherein the jaws include electrodes for applying energy to tissue to treat (e.g., cauterize, ablate, fuse, or cut) the tissue. In addition, the features of the presently described surgical instruments may be readily adapted for use in other types of surgical staplers, such as circular staplers, linear and/or purse string staplers and the like.

The surgical stapling and cutting instruments described herein may be minimally invasive (e.g., laparoscopic) instruments or instruments used for open surgery. Additionally, the features of the presently described surgical stapling instruments may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

The devices described herein, or certain components of the devices, may also be incorporated into a variety of different surgical instruments, such as those described in commonly assigned, co-pending U.S. patent application Ser. No. 16/205,128, Ser. No. 16/427,427, Ser. No. 16/678,405, Ser. No. 16/904,482, Ser. No. 17/081,088 and Ser. No. 17/084,981 and International Patent Nos. PCT/US2019/107646, PCT/US2019/019501, PCT/US2019/062344, PCT/US2020/54568, PCT/US2019/064861, PCT/US2019/062768, PCT/2020/025655, PCT/US2020/056979, PCT/2019/066513, PCT/US2020/020672, PCT/US2019/066530 and PCT/US2020/033481, the complete disclosures of which are incorporated by reference herein in their entirety for all purposes as if copied and pasted herein.

The term “coupler” as used herein means any device, component, system, assembly or other element that connects, combines, couples, conjoins, bonds, joins, unites, or links two or more components or devices together. The term “movably couples” means any such device component, system, assembly or other element that connects, combines, couples, conjoins, bonds, joins, unites, or links two or more components or devices together such that at least one of the components moves, pivots, rotates, translates, shifts, realigns, relocates or otherwise changes position, relative to at least one of the other components.

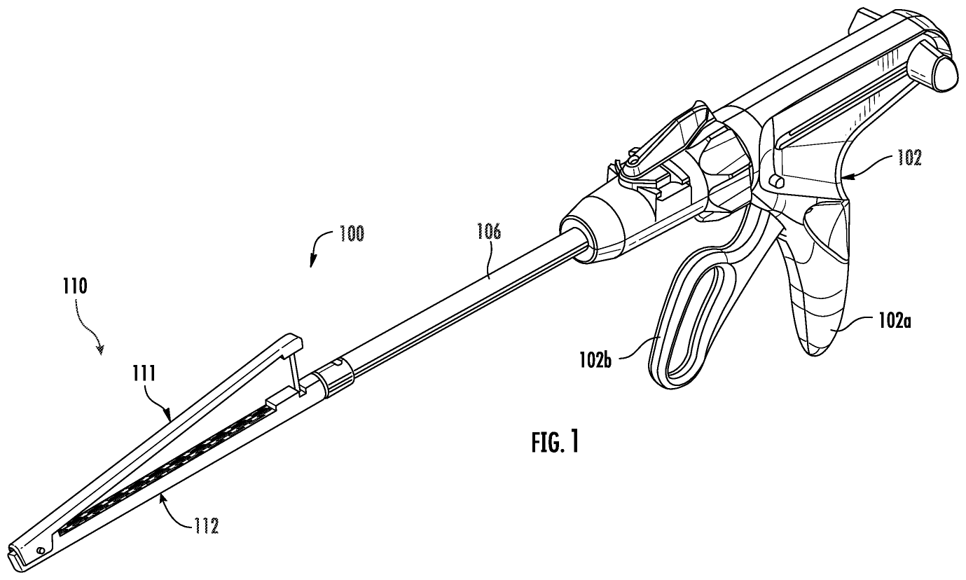

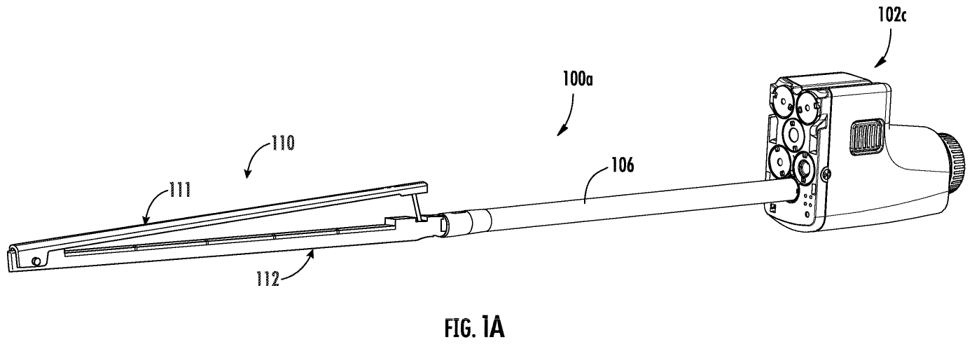

FIG. 1 is a perspective view of an illustrative surgical instrument 100 having a handle assembly 102, and an end effector 110 mounted on an elongated shaft 106. End effector 110 includes first and second jaws 111, 112. Handle assembly 102 includes a stationary handle 102a and a moveable handle 102b which serves as an actuator for surgical instrument 100.

FIG. 1A illustrates a surgical instrument 100a that includes a backend mechanism 102c instead of the handle assembly shown in FIG. 1. Backend mechanism 102c typically provides a mechanical coupling between the drive tendons, bands or cables of the instrument and the motorized axes of the mechanical interface of a drive system. Further details of known backend mechanisms and surgical systems are described, for example, in U.S. Pat. Nos. 8,597,280, 7,048,745, and 10,016,244. Each of these patents is hereby incorporated by reference in its entirety.

The input couplers may interface with, and be driven by, corresponding output couplers (not shown) of a telesurgical surgery system, such as the system disclosed in U.S Pub. No. 2014/0183244A1, the entire disclosure of which is incorporated by reference herein. The input couplers are drivingly coupled with one or more input members (not shown) that are disposed within the instrument shaft 106. The input members are drivingly coupled with the end effector 110. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety. Further details of known input couplers and surgical systems are described, for example, in U.S. Pat. Nos. 8,597,280, 7,048,745, and 10,016,244. Each of these patents is hereby incorporated by reference in its entirety for all purposes.

Actuation mechanisms of surgical instrument 100 may employ drive cables, rods or bands that are used in conjunction with a system of motors and pulleys. Powered surgical systems, including robotic surgical systems that utilize drive cables or bands connected to a system of motors and pulleys for various functions including opening and closing of jaws, as well as for movement and actuation of end effectors are well known. Further details of known drive cable and band surgical systems are described, for example, in U.S. Pat. Nos. 7,666,191, 8,271,230 and 9,050,119 and Publication No. WO 2020/252184, all of which are hereby incorporated by reference in their entireties. While described herein with respect to an instrument configured for use with a robotic surgical system, it should be understood that the actuation and drive assemblies described herein may be incorporated into manually actuated instruments, electro-mechanical powered instruments, or instruments actuated in any other way.

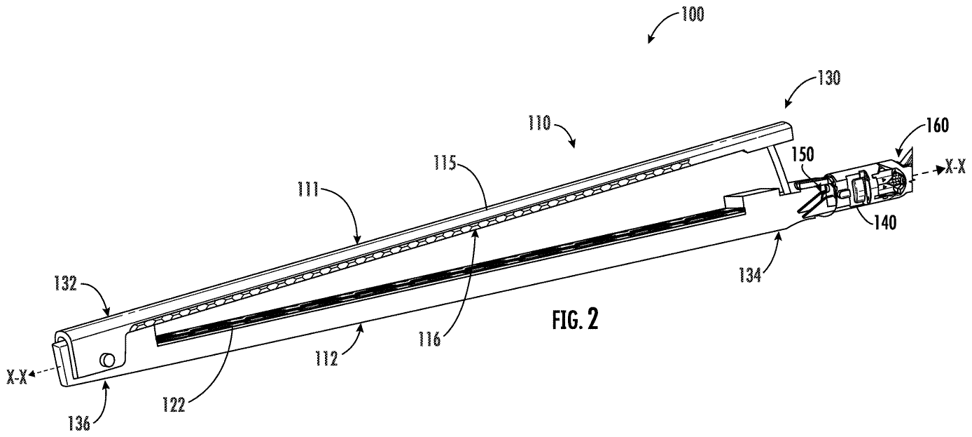

FIG. 2 shows the distal end portion of surgical instrument 100, including an end effector 110 defining a longitudinal axis X-X and having a first jaw 111, a second jaw 112, a clevis 140 for mounting jaws 111, 112 to the instrument, and an articulation mechanism, such as a wrist assembly 160. In certain embodiments, second jaw 112 is a movable jaw configured to move from an open position to a closed position relative to first jaw 111. In other embodiments, first jaw 111 is a movable jaw configured to move between open and closed positions relative to second jaw 112. In still other embodiments, both jaws 111, 112 are movable relative to each other.

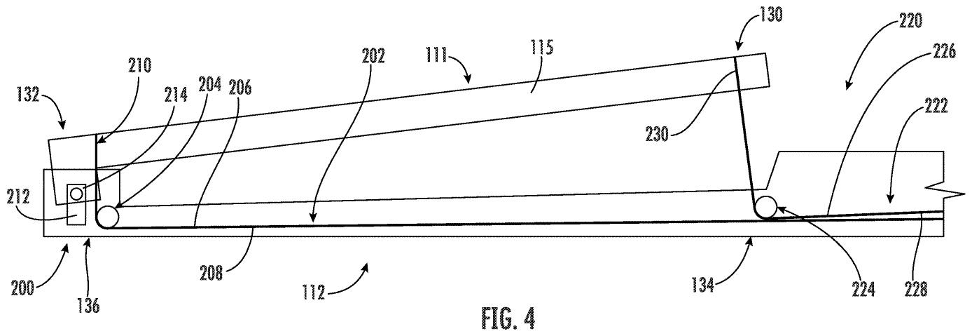

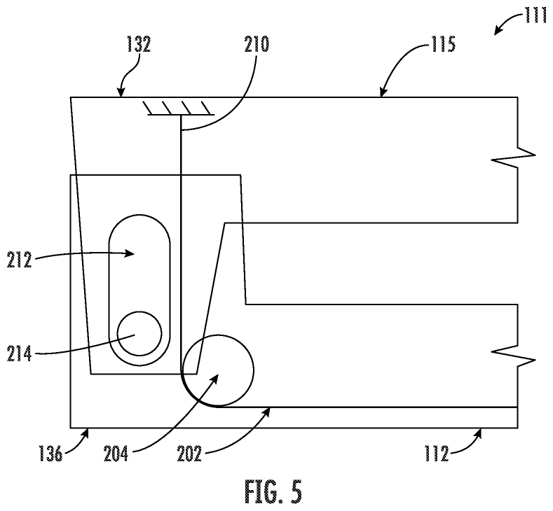

In the exemplary embodiment, first jaw 111 is a movable jaw 111 configured to move from an open position to a closed position relative to stationary jaw 112. In particular, movable jaw 111 has a proximal end portion 130 and a distal end portion 132 and second jaw 112 has a proximal end portion 134 and a distal end portion 136. As discussed in further detail below (see FIGS. 4 and 5), distal end portions 132, 136 of jaws 111, 112 are pivotally coupled to each other such that proximal end portion 130 of movable jaw 111 opens and closes relative to proximal end portion 134 of jaw 112 (which also adjusts the distance between proximal end portions 130, 134). In addition, the distance between distal end portions 132, 136 may be adjusted independently of the opening and closing of proximal end portions 130, 134.

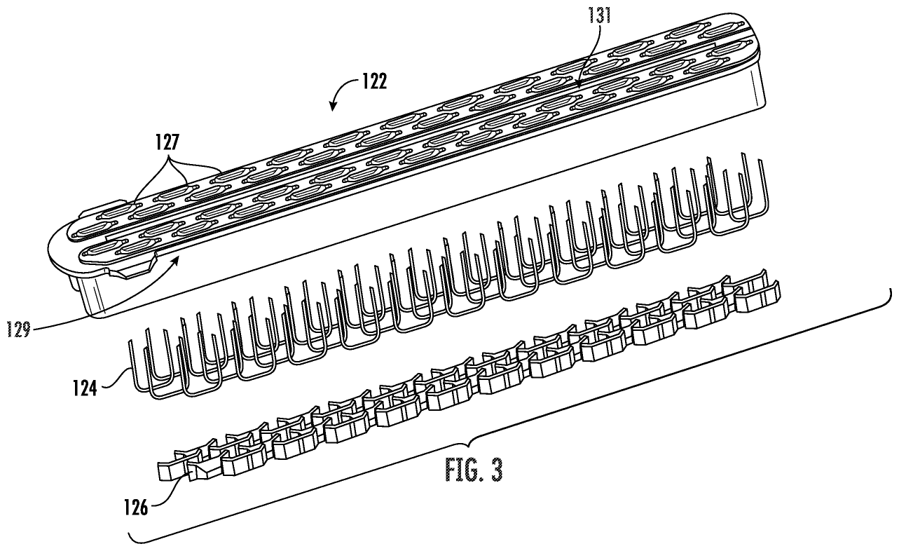

First jaw 111 includes an anvil 115 having staple-forming pockets 116. In the open position, an unused stapler cartridge 122 (sometimes referred to as a fresh or unfired reload) can be loaded into second jaw 112 and tissue may be positioned between the jaws 111, 112. In the closed position, jaws 111, 112 cooperate to clamp tissue such that stapler cartridge 122 and the anvil 115 are in close cooperative alignment. Staple forming pockets 116 are aligned with each other so as to cooperate to deform the first and second legs, respectively, of each staple 124 (see FIG. 3). Each forming pocket 116 is designed to receive a leg of the staple 124 and bend or deform the staple 124 such that the staple forms a suitable shape for securing tissue that is held between jaws 111, 112. Suitable shapes for deformed staples include B-shapes, D-shapes, M-shapes and the like.

In certain embodiments, the end effector 110 may be used, for example, to create a straight sleeve gastrectomy pouch in a stomach of a patient. In these embodiments, anvil 115 may be positionable on the anterior side of the stomach and the cartridge 122 on the posterior side of the stomach (or vice versa). The length of the anvil 115 and cartridge 122 may be sufficient for the anvil and cartridge to encircle the stomach. By way of example and not limitation, the length of each may measure from about 35 mm to about 350 mm.

As shown in FIG. 3, stapler cartridge 122 may include a plurality of staples 124 supported on corresponding staple drivers 126 provided within respective staple retention openings or pockets 127 formed in stapler cartridge 122. In one embodiment, cartridge 122 comprises a housing 129 having a central channel 131 for receiving a drive member 150 (shown in FIG. 2 and discussed in more detail below) and first and second staple receiving assemblies extending longitudinally on either side of central channel 131. Each staple receiving assembly comprises at least one linear row of staple pockets 127 for receiving staples 124. In some embodiments, the staple assemblies comprise two or more substantially parallel, linear rows of staple pockets 127. In the representative embodiment, cartridge 122 comprises three longitudinal rows of staple pockets 116, although it will be understood that cartridge 122 may comprise only one row, two rows, four rows, five rows, six rows or more. For example, in one embodiment, the staple pockets may be arranged in six longitudinal rows, with three rows of staple pockets positioned on a first side of a longitudinal axis of the cartridge 122 and three rows of staple pockets positioned on a second side of the longitudinal axis.

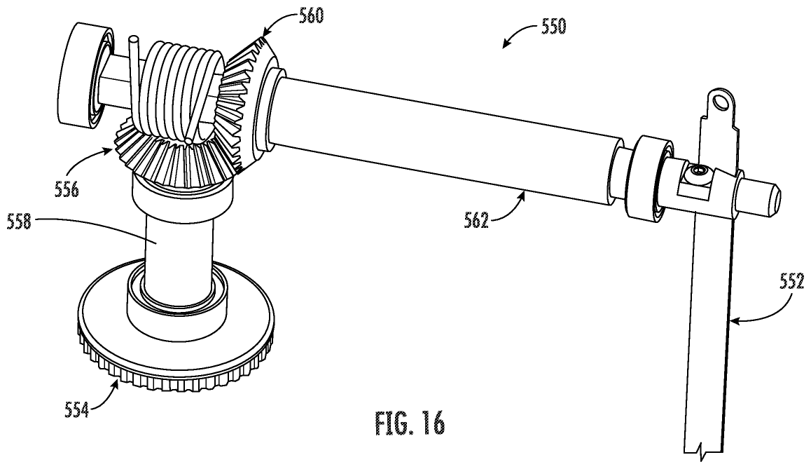

Referring now to FIGS. 4 and 5, one embodiment of proximal and distal couplers for adjusting the distance between the proximal and distal ends of jaws 111, 112 will now be described. In this embodiment, a distal coupler 200 includes a tension band 202, a pulley 204 and a proximal drive member (discussed below in reference to FIG. 16) for moving tension band 202 along a surface of pulley 204. Tension band 202 comprises a planar first surface 206 in contact with pulley 204, a planar second surface 208 opposite first surface 206 and a distal end 210 coupled to distal end portion 132 of jaw 111 (or anvil 115). Tension band 202 extends through a channel in jaw 112 or staple cartridge 122, through shaft 106 to a proximal end of instrument 100 (either to backend mechanism 102c or handle 102, see FIGS. 1 and 1A).

Jaw 111 further includes a slot 212 and anvil 115 includes a slot pin 214 (or vice versa) that slides through slot 212 to allow jaw 112 to move relative to jaw 111 and adjust the distance between distal end portions 132, 136 of the jaws 111, 112 from a fully open position, wherein the jaws 111, 112 define the largest gap therebetween, to a substantially closed position, wherein the jaws 111, 112 define a smaller gap therebetween. Slot 212 extends transversely to longitudinal axis X-X. In a preferred embodiment, slot 212 extends perpendicularly to longitudinal axis X-X so that slot pin 214 moves vertically relative to its orientation in FIG. 5.

In embodiments, distal coupler 200 limits a range of distance between the distal ends of anvil 115 and staple cartridge 122 between a minimum distance and a maximum distance. This range may be limited mechanically (i.e., by mechanically limiting the range of travel of band 202) or through a control system coupled to distal coupler 200 or to the proximal drive components for coupler 200. This range may be set, for example, based on the maximum and minimum distances that allow for adequate staple formation through a particular type of tissue once the staples have been driven against anvil 115 by drive member 150.

Distal coupler 200 may further include a spring (not shown) that biases jaws 111, 112 into either of the open or closed positions. In an exemplary embodiment, the spring biases jaws 111, 112 into the open position and application of a tension force on tension band 202 operates to pull the jaws 111, 112 closer together to adjust the gap therebetween.

Instrument 100 further includes a proximal coupler 220 that includes a tension band 222, a pulley 224 and a proximal drive member (discussed below) for moving tension band 222 along a surface of pulley 224. Tension band 222 comprises a planar first surface 226 in contact with pulley 224, a planar second surface 228 opposite first surface 226 and a distal end 230 coupled to proximal end portion 130 of anvil 115. Tension band 222 extends through a proximal end 134 of end effector 110, through shaft 106 to a proximal end of instrument 100 (either to backend mechanism 102c or handle 102, see FIGS. 1 and 1A).

In certain embodiments, distal end portions 132, 136 of jaws 111, 112 are pivotally coupled to each other by a hinge or other suitable mechanism such that proximal end portions 130, 134 may be pivoted between a fully open position and a substantially closed position. In certain embodiments, jaws 111, 112 are substantially parallel to each other in the substantially closed position. In embodiments, proximal coupler 200 limits a range of distance between the proximal ends of anvil 115 and staple cartridge 122 between a minimum distance and a maximum distance. This ranges may be set, for example, based on the maximum and minimum distances that allow for adequate staple formation through a particular type of tissue once the staples have been driven against anvil 115 by drive member 150.

Proximal coupler 200 may further include a spring (not shown) that biases jaws into either of the open or closed positions. In an exemplary embodiment, the spring biases jaws 111, 112 into the open position and application of a tension force on tension band 232 operates to pull the jaws 111, 112 closer together to pivot the jaws closed and to adjust the gap therebetween.

Bands 202, 222 (and any of the bands described herein) can have any suitable shape. For example, although FIGS. 4 and 5 show bands 202, 222 as having a rectangular cross-sectional shape, the bands may have other shapes, such as trapezoidal, triangular, curved, crowned and the like. Bands 202, 222 may be constructed of any suitable materials. In some embodiments, bands 202, 222 form a single, unitary band. In other embodiments, bands 202, 22 can be constructed from a series of laminates that are bonded together (e.g., via an adhesive or any other suitable method). The laminates may comprise any suitable material, such as tungsten, steel, polymers or the like.

In certain embodiments, tension may be applied to bands 202, 222 such that bands 202, 222 advance substantially in the proximal direction through end effector 110 and/or shaft 106 of instrument. Since bands 202, 222 are in contact with pulleys 204, 224, the distal portion of each band 202, 222 moves substantially perpendicular to end effector to pull distal and/or proximal end portions 130, 132 of anvil 115 towards jaw 111. In certain embodiments, compression may be applied to bands 202, 222 to move them in the opposite direction to push proximal and distal end portions 132, 136 away from each other. Alternatively, the tension may be reduced or removed altogether to allow the springs to automatically push distal end portions 132, 136 apart.

In certain embodiments, operation of tension bands 202, 222 may be entirely manual, i.e., proximal handle 102 or another user interface (not shown) coupled to backend mechanism 102c may include one or more actuators that allow the user to apply tension to bands 202, 222 to manually adjust the distances between the distal and proximal end portions of jaws 111, 112. In other embodiments (discussed in more detail below), operation of tension bands 202, 222 may be automatically controlled by one or more controllers (not shown) that are coupled to tension bands 202, 222 in handle 102 or backend mechanism 102c. In these embodiments, the tension applied to bands 202, 222 may be adjusted based on, for example, the torque or force required to move bands 202, 222 and consequently the torque or force applied by tissue onto jaws 111, 112 as jaws 111, 112 are moved closer together. In yet another embodiment, operation of tension bands 111, 112 may be a combination of manual and automatic controls. For example, manual adjustment of the tension on bands 111, 112 may be permitted by one or more controllers within a certain range of torque or force that is applied to jaws 111, 112 (discussed in more detail below).

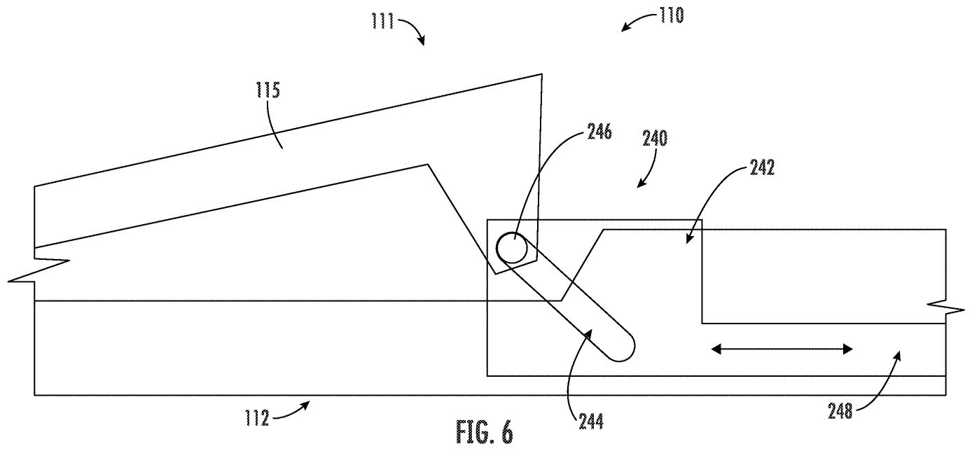

Referring now to FIG. 6, an alternative embodiment of a proximal coupler 240 for instrument 100 will now be described. As shown, proximal coupler 240 comprises a drive element 242 having a slot 244 that extends at a transverse angle relative to the longitudinal axis of end effector 110. Upper jaw 112 includes a slot pin 246 that slides through angled slot 244 such that distal movement of drive element 242 causes slot pin 246 to slide downwards through angled slot 244, thereby causing jaw 112 to move towards the closed position and reduce the gap between proximal end portions 130, 134 of jaws 111, 112. Proximal movement of drive element 242 causes slot pin 246 to slide upwards through angled slot 244, thereby causing jaw 112 to move towards the open position and increase the gap between proximal ends 130, 134 of jaws 111, 112.

Drive element 242 includes an elongate member 248 that extends proximally to a proximal end of shaft 106 and is movable within shaft 106 in the longitudinal direction. In some embodiments, drive element 242 may be coupled to drive member 150 such that it moves distally as drive member 150 is driven distally, thereby automatically closing the jaws 111, 112 as drive member 150 moves distally through end effector 110. In other embodiments, drive element 242 is not coupled to drive member 150 and can be independently actuated in a similar fashion as bands 202, 222.

In certain embodiments, instrument 100 may include proximal coupler 240 and a distal coupler 200 as shown in FIGS. 4 and 5 to allow for independent adjustment of the distances or gaps between both the proximal and distal ends of the jaws. In other embodiments, instrument 100 may include only proximal coupler 240 and the distal ends of jaws 111, 112 will be pivotally coupled to each other to allow for opening and closing of the jaws.

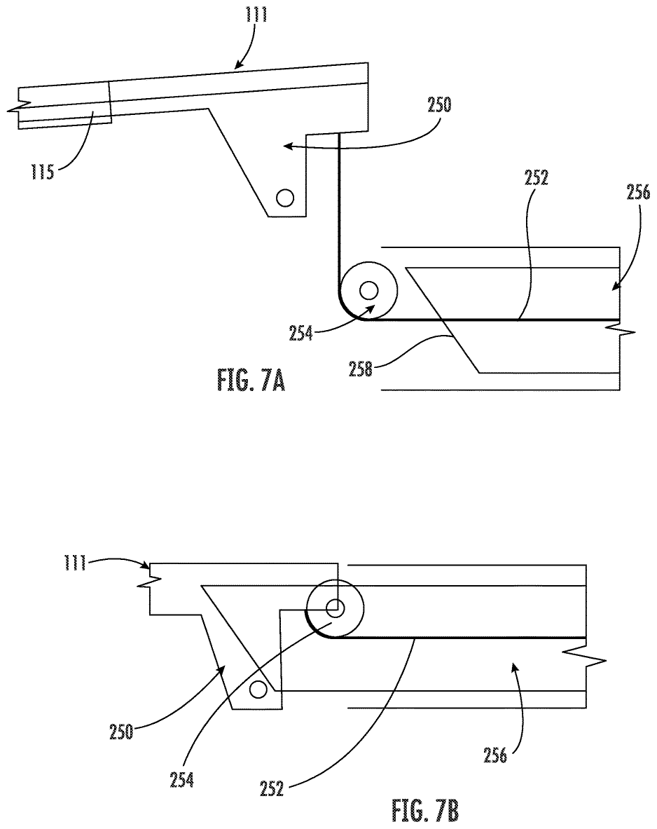

Referring now to FIGS. 7A and 7B, another embodiment of a proximal coupler will now be described. As shown, the coupler comprises a substantially planar band 252 that extends through shaft 106 to the proximal ends of jaws 111, 112. Band 252 has a distal end attached to anvil 115 and, is in contact with the surface of a pulley 254. As in previous embodiments, the proximal end of band 252 is coupled to a drive element configured to push/pull band 252 in a longitudinal direction to adjust the distance between the proximal ends of the jaws.

In this embodiment, the articulation assembly further includes a drive member 256 having a tapered distal surface 258. Drive member 256 may be the same element as drive member 150 that operates to fire the staples (discussed below), or it may be a separate element that is driven independently of drive member 150. In certain embodiments, tapered distal surface 258 of drive member 256 is configured to engage a surface of pulley 254 to rotate pulley and advance band 252. For example, distal movement of driver member 256 may cause pulley to rotate such that band 252 moves proximally, thereby moving the proximal end of jaw 111 towards jaw 112. In other embodiments, a proximal drive element may operate to move drive member 256 distally simultaneously with the proximal movement of band 252. In both embodiments, drive member 256 engages jaws 111, 112 as it moves distally to ensure that jaws 111, 112 are substantially parallel to each other prior to firing of staples (discussed below).

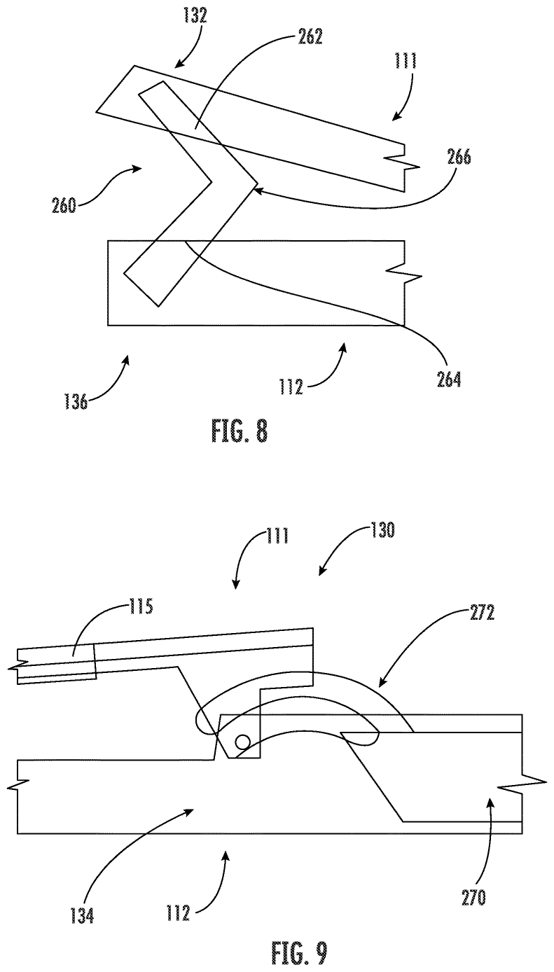

Referring now to FIG. 8, another embodiment of a distal coupler will now be described. As shown, the distal coupler comprises a link assembly 260 having a first link 262 coupled to distal end 130 of jaw 111 and a second link 264 coupled to distal end 136 of jaw 112. First and second links 262, 264 are coupled to each other at a pivot point or central portion 166 of link assembly 260. In embodiments, links 262, 264 may be pivotally coupled to each other such that longitudinal movement of central portion 166 pivots links 262, 264 and opens and closes the jaws 111, 112. In other embodiments, links 262, 264 are rigidly coupled to each other at central portion 266 such that longitudinal movement of portion 266 moves the entire link assembly 260 in a proximal or distal direction to open and close distal ends 130, 136 of the jaws.

Referring now to FIG. 9, another embodiment of a proximal coupler will now be described. As shown, the linage comprises a drive member 270, which may be the same element as drive member 150 that operates to fire the staples (discussed below), or it may be a separate element that is driven independently of drive member 150. Drive element 270 is coupled to a link assembly 272:

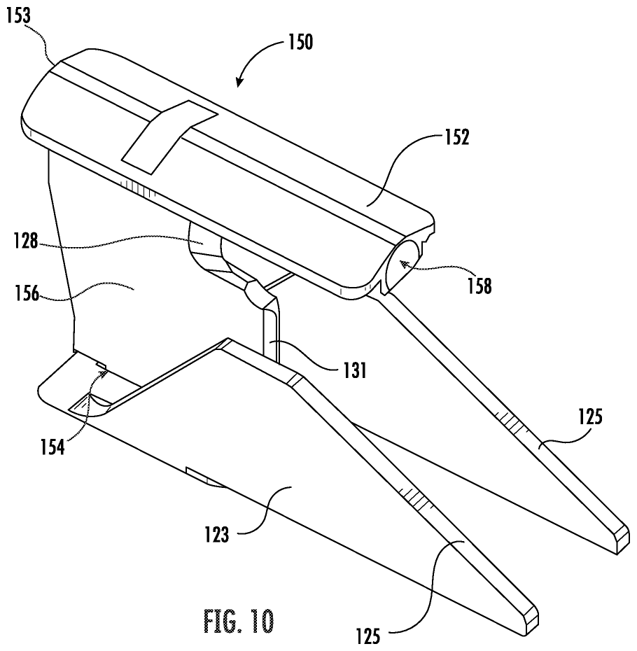

Referring now to FIG. 10A, surgical instrument 100 may also include a drive member 150 configured to translate through the end effector 110. Drive member 150 may be any structure capable of pushing at least one of a shuttle or a knife of a surgical stapling instrument with the necessary force to effectively sever or staple human tissue. Drive member 150 may be an I-beam, an E-beam, or any other type of drive member capable of performing similar functions. Drive member 150 is movably supported on the surgical stapling instrument 100 such that it may pass through cartridge 122 and upper jaw 111 and lower jaw 112 when the surgical stapling instrument is fired (e.g., actuated).

In the embodiment of FIG. 10A, the instrument will also include a shuttle (not shown) that is separate from drive member 150. The shuttle may, for example, be included within stapler cartridge 122 as a separate component and includes one or more inclined portions that sequentially act on stapler drivers 126 upon distal or proximal movement of drive member 150 (see, for example, the inclined portions 125 on the integral shuttle 123 shown in FIG. 10B).

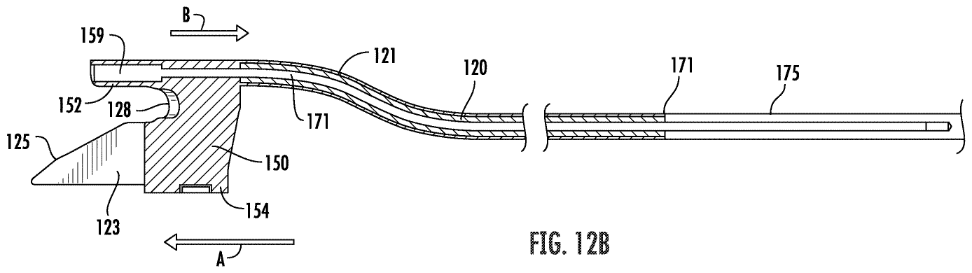

In a representative embodiment, drive member 150 may include an upper protrusion or shoe 152, a lower protrusion or shoe 154, and a central portion 156 connecting upper and lower shoes 152, 154. Upper shoe 152 of drive member 150 is substantially aligned with and translates through a channel (not shown) in jaw 111, while lower shoe 154 of drive member 150 is substantially aligned with and translates through a channel (not shown) and below jaw 112. A bore 158 is formed through central portion 156 to receive a drive cable 171 (see FIG. 12A) as will be described in more detail below. A proximal surface of drive member 150 is configured to be engaged by a coil 120 (see FIG. 12A) of an actuation assembly such that coil 120 may apply force to drive member 150 to advance drive member 150 distally, i.e., in the direction of arrow “A” in FIG. 12B. A knife 128 may be formed on drive member 150 along the distal edge between upper shoe 152 and central portion 156.

Upon actuation of the surgical instrument, drive member 150 is advanced distally through end effector 110 to advance shuttle 123 and knife 128 distally through cartridge 122 to staple and cut tissue grasped between jaws 111, 112. In an alternative embodiment, drive member 150 may be advanced proximally from a distal end of end effector 110 to advance shuttle 123 and knife 128 proximally through cartridge 122.

In some embodiments, drive member 150 may work in concert with the proximal and distal couplers discussed above to move jaws 111, 112 from the open position to the closed position. In other embodiments, the jaws 111, 112 may already be moved into a closed or substantially closed position before drive member 150 is actuated to move distally. In certain embodiments, the proximal and distal couplers are first actuated to move jaws 111, 112 into an optimal position relative to the thickness of tissue therebetween. After this occurs, distal translation of drive member 150 may further compress jaws 111, 112 together to clamp tissue during the stapling and dissection operations.

It should be noted that the instrument is not limited to the configuration wherein the drive member 150, when “fired”, is translated distally to act on staple drivers 126 and move staples 124 into deforming contact with anvil 115. In certain embodiments, drive member 150 may be located within the proximal end portion of end effector 110 and configured to, when “fired”, translate proximally to thereby act on staple drivers 126 and move staples 124 into deforming contact with anvil 115.

FIG. 10B illustrates another embodiment of drive member 150A that includes a shuttle 123 integrally formed thereon. Shuttle 123 comprises an inclined distal or proximal portion 125 that sequentially acts on staple drivers 126 upon distal or proximal movement of the drive member 150A, camming staple drivers 126 upwardly, thereby moving staples 124 into deforming contact with anvil 115. In certain embodiments, shuttle 123 may be included within stapler cartridge 122 as a separate component.

In a representative embodiment, drive member 150A may include an upper protrusion or shoe 152, a lower protrusion or shoe 154, and a central portion 156 connecting upper and lower shoes 152, 154. Upper shoe 152 of drive member 150A is substantially aligned with and translates through a channel (not shown) in jaw 111, while lower shoe 154 of drive member 150A is substantially aligned with and translates through a channel (not shown) and below jaw 112. A bore 158 is formed through upper shoe 152 to receive a drive cable 171 (see FIG. 12A) as will be described in more detail below. Proximal surface 153 of upper shoe 152 is configured to be engaged by a coil 120 (see FIG. 12A) of an actuation assembly such that coil 120 may apply force to upper shoe 152 to advance drive member 150 distally, i.e., in the direction of arrow “A” in FIG. 12B. A knife 128 may be formed on drive member 150A along the distal edge between upper shoe 152 and central portion 156. In embodiments, inclined distal portions 125 may be formed on either side of drive member 150A.

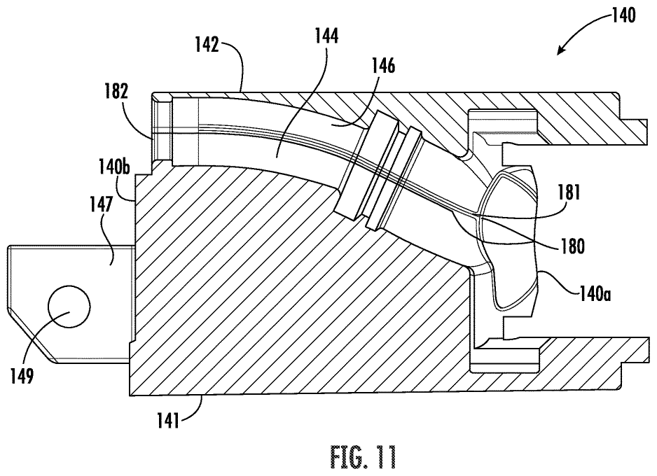

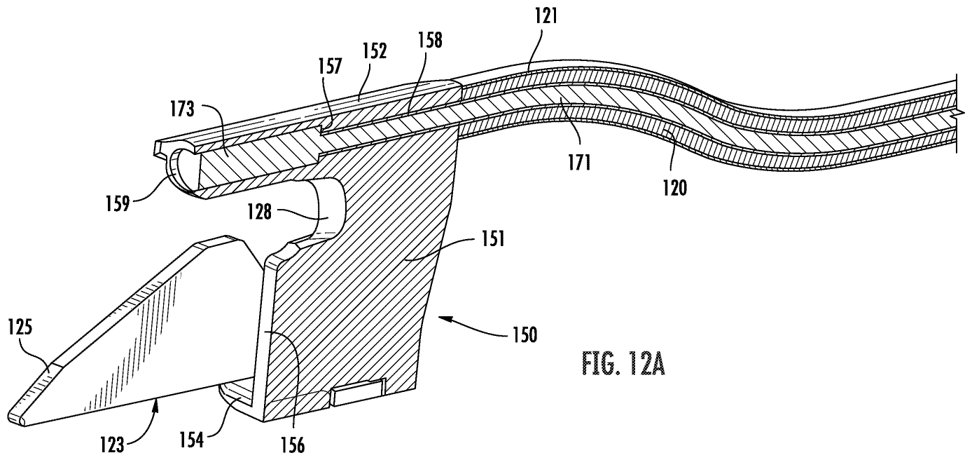

Referring now to FIG. 11, jaws 111, 112 may be attached to surgical instrument 100 via a clevis 140 or other suitable coupling element. In one representative embodiment designed to work with drive member 150A in FIG. 10B, clevis 140 includes a proximal surface 140a and a distal surface 140b. Clevis 140 further includes upper clevis portion 142 and lower clevis portion 141 that cooperate when assembled to form a protrusion configured to engage tabs of jaw 111 to securely mount jaw 111 in a fixed position on instrument 100. Lower clevis portion 141 includes a pair of distally extending arms 147 for supporting movable jaw 112. Arms 147 include opening 149 for receiving a pivot pin (not shown) defining a pivot axis around which jaw 112 pivots as described in more detail below.

Lower clevis portion 141 also includes ramped groove 144 configured to guide a portion of an actuation coil 120 (see FIG. 12A) emerging from wrist 160. Upper clevis portion 142 includes a complementary shaped ramped groove 146 that cooperates with ramped groove 144 of lower clevis portion 141 to form an enclosed channel 180 that guides coil 120 as it jogs upwards from wrist 160 towards distal surface 157 of upper shoe 152 of drive member 150A. In embodiments, channel 180 may include a first end 181 at a central portion of proximal surface 140a and a second end 182 at a peripheral portion of distal surface 140b. In embodiments, enclosed channel 180 may be substantially “S” shaped. Although shown as a two-part clevis, it should be understood that the clevis may be a unitary structure formed, for example, by molding, machining, 3-D printing, or the like.

Referring now to FIGS. 12A and 12B, a representative actuation assembly for drive member 150A includes a drive cable 171, a coil 120, a sheath 121 surrounding coil 120, and a drive rod 175. Drive cable 171 includes an enlarged distal end 173. Upper shoe 152 of drive member 150A includes a bore 158 into which drive cable 171 is routed (note that in the embodiment of FIG. 10A, the bore 158 is positioned within central portion 156).

When assembling illustrative surgical instrument 100, coil 120 and a protective sheath 121 are slipped over the free end of drive cable 171. The free end of drive cable 171 is attached to a drive rod 175 securing coil 120 and the protective sheath 121 between drive member 150 and drive rod 175 as seen in FIG. 12B. Sheath 121 may function to promote stability, smooth movement, and prevent buckling upon actuation of surgical instrument 100. Sheath 121 may be made from polyimide, or any other suitable material having the requisite strength requirements such as various reinforced plastics, a nickel titanium alloy such as NITINOL™, poly para-phenyleneterphtalamide materials such as KEVLAR™ commercially available from DuPont. Other suitable materials may be envisioned by those of skill in the art.

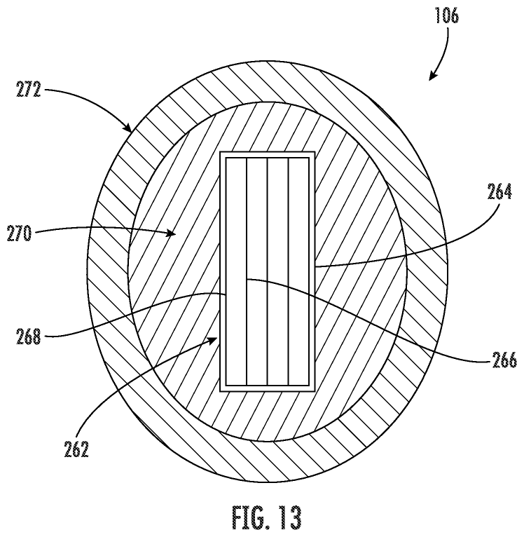

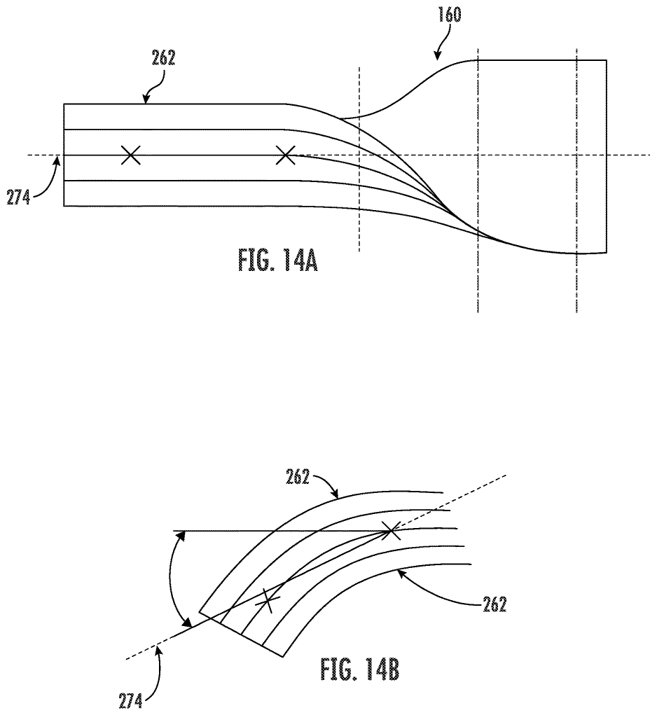

Enlarged distal end 173 of drive cable 171 resides within an enlarged distal portion 159 of bore 158 in upper shoe 152 of body 150, such that the proximal face 157 of enlarged distal end 173 may apply a retraction force on upper shoe 152 when the drive cable 171 is pulled proximally, i.e., in the direction of arrow “B” in FIG. 12B. Drive rod 175 is operationally connected to an actuator (e.g., movable handle 102b), which allows distal translation and proximal retraction of actuation assembly 190. Those skilled in the art will recognize that in a manually actuated instrument, the actuator may be a movable handle, such as moveable handle 102b shown in FIG. 1; in a powered instrument the actuator may be a button (not shown) that causes a motor to act on the drive rod; and in a robotic system, the actuator may be a control device such as the control devices described below in connection with FIGS. 13-16. Any suitable backend actuation mechanism for driving the components of the surgical stapling instrument may be used. For additional details relating to exemplary actuation mechanisms using push/pull drive cables see, e.g., commonly owned International Application WO 2018/049217, the disclosure of which is hereby incorporated by reference in its entirety.

During actuation of illustrative surgical instrument 100, drive rod 175 applies force to coil 120, thereby causing coil 120 to apply force to upper shoe 152 (or central portion 156) of drive member 150, translating it distally (i.e., in the direction of arrow “A” in FIG. 12B) initially closing jaws 111,112 and then ejecting staples 124 from cartridge 122 to staple tissue. After stapling is complete, drive rod 175 applies a force in the proximal direction to effect retraction of the drive member. During retraction, enlarged distal end 173 of drive cable 171 is obstructed by wall 157 of enlarged portion 159 of bore 158, causing drive cable 171 to apply force to upper shoe 152 (or central portion 156) of the drive member, thereby translating the drive member in the proximal direction. In certain embodiments, the surgical instrument may be designed such that the drive member 150 is not retracted in the proximal direction after the staples have been fired. One of ordinary skill in the art will appreciate that drive member 150, drive cable 171, and drive rod 175 all move in unison and remain in the same relative position to each other.

In embodiments, surgical instruments may alternatively include switches designed to be engaged by an inclined distal portion of a drive member for purposes of engaging a lockout assembly, providing for reload recognition, or both, as described in International Patent Application Nos. PCT/US2019/66513 and PCT//US2019/66530, both filed on Dec. 16, 2019, the entire disclosures of which are incorporated herein by reference.

End effector 110 may be articulated in multiple directions by one or more articulation assemblies. In certain embodiments, the articulation assemblies may include articulation elements in wrist 160, although other articulation mechanisms are contemplated. In certain embodiments, end effector 110 may be articulated to rotate about the longitudinal axis of shaft 106. In other embodiments, end effector 110 may be articulated to rotate about one or more axes that are substantially perpendicular to shaft 106. In one such embodiment, end effector 110 may be rotated about a yaw axis and/or a pitch axis relative to shaft 106.

Referring now to FIGS. 13, 14A and 14B, one embodiment of an articulation assembly 260 will now be described. As shown, articulation assembly 260 comprises a plurality of bands 262 extending through an internal channel 264 in shaft 106 and having proximal ends coupled to a suitable drive element (not shown). Internal channel 262 is preferably contained within an outer sheath 270 and an outer tube 272 of shaft 106. In some embodiments, bands 262 may be coupled to a portion of drive member 150. This configuration provides some additional rigidity to bands 262 to, for example, inhibit buckling in the push direction.

Bands 262 each include a first planar surface 266 and a second planar surface 268 opposite the first surface 266. In an exemplary embodiment, bands 260 are oriented such that surfaces 266, 268 are substantially perpendicular to the longitudinal axis of shaft 106. Bands may be 262 are constructed from a series of laminates that are bonded together (e.g., via an adhesive or any other suitable method). The laminates may comprise any suitable material, such as tungsten, steel, polymers or the like.

As shown in FIGS. 14A and 14B, bands 262 are twisted within wrist 160 by at least about 45 degrees, preferably about 90 degrees, relative to the direction of bands 262 within shaft 106. A proximal drive element (not shown) is configured to push/pull bands 262 within shaft 106, which then causes the portion of bands 262 within wrist 160 to rotate end effector 110 relative to shaft 106. In certain embodiments, bands 262 are designed to rotate end effector 110 about one axis substantially perpendicular to the longitudinal axis 274 of shaft 106. In other embodiments, bands 262 may rotate end effector about two different axes that are substantially perpendicular to the longitudinal axis of shaft 106 and substantially perpendicular to each other (i.e., yaw and pitch axes).

Other articulation mechanisms within the purview of those skilled in the art may substitute for bands 262 and/or wrist 160. One suitable articulation mechanism is described for example in U.S. Publication No. 2015/0250530, the disclosure of which is hereby incorporated by reference in its entirety.

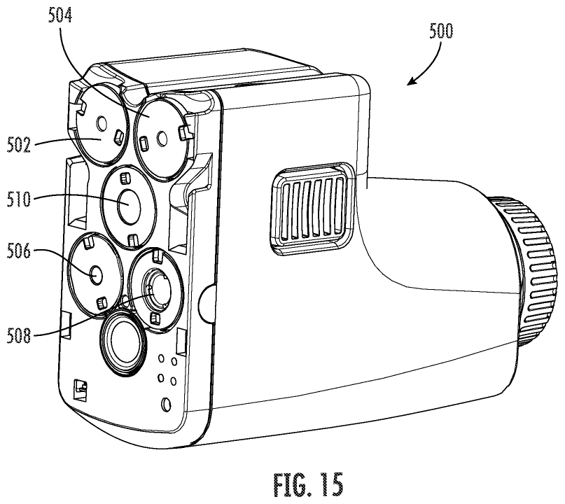

Referring now to FIG. 15, one embodiment of a representative backend mechanism 500 for instrument 100 will now be described. Backend mechanism 500 includes a housing 501 and a series of rotary inputs 502, 504, 506 and 508 that control movement of the bands described above to manipulate the end effector of the instrument. In addition, backend mechanism includes a rotary input 510 for actuating drive member 150 to provide for dissection and stapling functions of the instrument.

In one embodiment, rotary inputs 502, 504 produce movement of bands 202, 222 (FIGS. 4 and 5) to control the distances or gaps between proximal and distal ends of anvil and staple cartridge. Rotary inputs 506, 508 controls bands 262 to produce the desired movement (pitch, yaw and/or roll) at the wrist assembly. Rotary input 510 controls drive member 150 to advance drive member 150 through end effector 110 to drive staples against anvil and dissect tissue, as discussed above.

Specifically, backend mechanism 500 includes components and controls to move bands 202, 222 in a proximal direction (i.e., to pull in bands 202, 222) to decrease the gap between the proximal and distal ends of the jaws. These components and controls may also allow for the distal movement (i.e., releasing or “paying out”) of bands 202, 222 to allow the springs in articulation assemblies 200, 220 to force bands to move in the distal direction to increase the gap between the proximal and distal ends of the jaws. Similarly, backend mechanism 500 includes components and controls to move bands 262 in the proximal and distal directions to rotate end effector 110 about the longitudinal axis of shaft 106 (i.e., roll) and/or one or more axes perpendicular to shaft 106 (i.e., pitch and/or yaw).

The backend mechanisms may include, for example, a gimbal, a lever, or any other suitable mechanism to directly pull (or release) a proximal end portion any of the bands. For example, in some embodiments, backend mechanism 500 can include the backend assemblies or components described in US Patent Application Publication No. US 2015/0047454, filed Aug. 15, 2014, or U.S. Pat. No. 6,817,942, filed Jun. 28, 2001, each of which is incorporated herein by reference in its entirety for all purposes. In other embodiments, backend mechanism 500 may include a capstan or other motor-driven roller that rotates or “winds” a portion of any of the bands to produce the desired band movement. For example, in some embodiments, backend mechanism 500 may include any of the backend assemblies or components described in U.S. Pat. No. 9,204,923, filed Jul. 16, 2008, which is incorporated herein by reference in its entirety for all purposes.

FIG. 16 illustrates one embodiment of a proximal drive assembly 550 within backend mechanism 500 for producing movement of a band 552 within the instrument. As shown, drive assembly 550 comprises a rotary input 554 that may be actuated manually by the user, or remotely via a robotic control system. Rotary input 554 is coupled to a first gear 556 by a shaft 558. First gear 556 is coupled to a second gear 560, which is, in turn, coupled to a rod 562. Rotation of input 554 causes rotation of shaft 558 and first gear 556. First gear 556 imparts rotation of second gear 560 and rod 562. As shown a band 552 is coupled to rod 562 and configured to wind up (i.e., move towards rod 562) or “pay out” (i.e., move away from rod 562) upon rotation of rod 562.

The surgical instruments described herein may be coupled to a proximal control system that monitors and controls the couplers for adjusting the gaps between the distal and proximal ends of the jaws, the drive member that fires the staples and dissects tissue and/or the articulation mechanisms for rotating the end effector relative to the shaft of the instrument. This control system may be a manual control system with user interfaces that allow the user to control each of the functions of the instrument, or it may be an automatic control system that monitors and controls these functions. In some embodiments, the control system is a combination of manual and automatic that allows the user to adjust or control certain functions, while automatically limiting those functions within certain ranges or parameters.

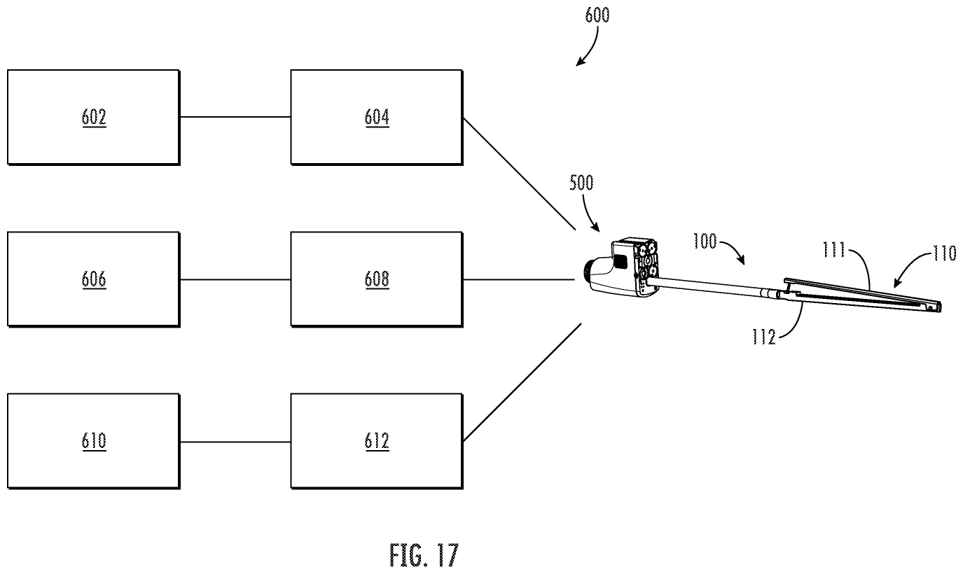

FIG. 17 schematically illustrates a control system 600 detecting the position of a surgical instrument 100 and for utilizing that information to assist in surgical procedures. Control system 600 includes a first controller 602 configured to detect a torque and/or force applied to the proximal ends of the anvil and the cartridge and/or to the proximal coupler that adjusts the gap between these proximal ends. First controller 602 may be coupled to one or more sensors (not shown) positioned within the end effector for detecting the torques and/or forces applied thereto. The first controller 602 is coupled to a drive assembly 604, such as one of the drive assemblies described herein, that is configured to adjust the distance between the proximal ends of the anvil and the cartridge. First controller 602 controls the drive assembly 604 such that the torque or force remains within a first range. Drive assembly 604 may be positioned within backend assembly 500 of surgical instrument 100.

In embodiments, the torque and/or force is associated with one or more forces applied to the anvil and the cartridge by tissue disposed therebetween. Controller 602 may be configured to maintain the distances of the proximal and distal ends of the jaws 111, 112 within ranges based on these forces or torques. In one such embodiment, for example, the ranges may be based on a minimum distance representing a threshold force applied by the tissue to the jaws such that the jaws do not compress the tissue too much to effectively apply staples thereto. The ranges may also, for example, be based on a maximum distance representing a threshold and/or zero amount of force applied by the tissue to the jaws such that the jaws are not separated so far from each other to be unable to effectively apply staples to the tissue.

In certain embodiments, first controller 602 is configured to limit operation of the drive assembly 604 such that the proximal ends of the anvil and cartridge cannot be moved into positions that create a torque or force outside of the prescribed range. In other embodiments, the first controller is configured to control drive assembly 604 to move the proximal ends of the anvil and cartridge into positions that create a torque or force within the prescribed range.

Control system 600 includes a second controller 606 configured to detect a torque and/or force applied to the distal ends of the anvil and the cartridge. Second controller 606 may be coupled to one or more sensors (not shown) on the end effector for detecting the torques and/or forces applied thereto. The second controller 606 is coupled to a drive assembly 608, such as one of the drive assemblies described herein, that is configured to adjust the distance between said distal ends. Second controller 606 controls the drive assembly 608 such that the torque or force remains within a first range and may be located within backend mechanism 500.

In certain embodiments, second controller 606 is configured to limit operation of the drive assembly 608 such that the distal ends of the anvil and cartridge cannot be moved into positions that create a torque or force outside of the prescribed range. In other embodiments, second controller 606 is configured to control drive assembly 608 to move the distal ends of the anvil and cartridge into positions that create a torque or force within the prescribed range.

First and second controllers 602, 606 may be separate from each other, or part of the same controller. In some embodiments, controllers 602, 606 are part of a robotic control system coupled to the surgical instrument.

System 600 may include a third controller 610 coupled to an actuator 612 for firing drive member 150 (see FIG. 2) and configured to inhibit or prevent the advancement of drive member 150 when either of the first or second torques are outside of the first and second ranges, respectively. Third controller 610 may be independent of first and/or second controllers 602, 606, or the controllers may cooperate with each other as part of a single control system.

First, second and third controllers 602, 606, 610 can include one or more processors (e.g., microprocessor, microchip, or application-specific integrated circuit), one or more memory devices (e.g., random-access memory and/or read-only memory), and I/O interface and/or a communication interface. The processors may include one or more computer-readable storage devices and/or software applications that store program instructions that allow the processor(s) to compare the detected torque or force with the prescribed range. The I/O devices can include one or more devices that enable the user to interact with the system (e.g., a user interface). The I/O devices can include, for example, a touchscreen display, a keypad, one or more selectors, one or more indicators.

Although described as a processor, it is to be appreciated that controllers 602, 606, 610 may be implemented in practice by any combination of hardware, software and firmware. Also, their functions as described herein may be performed by one unit, or divided up among different components, each of which may be implemented in turn by any combination of hardware, software and firmware.

The sensors may include any suitable sensors for detecting force and/or torque. In one embodiment, the sensors include fiber optic bend sensors, such as Fiber Bragg Gratings (FBG) for providing strain measurements in the jaws, the tension bands and/or other components of the surgical instrument. Various systems and methods for monitoring the shape and relative position of an optical fiber in three dimensions are described in U.S. patent application publication no. 2006/0013523, filed on Jul. 13, 2005, and U.S. Pat. No. 6,389,187, filed on Jun. 17, 1998, the completed disclosures of which are incorporated herein by reference for all purposes.

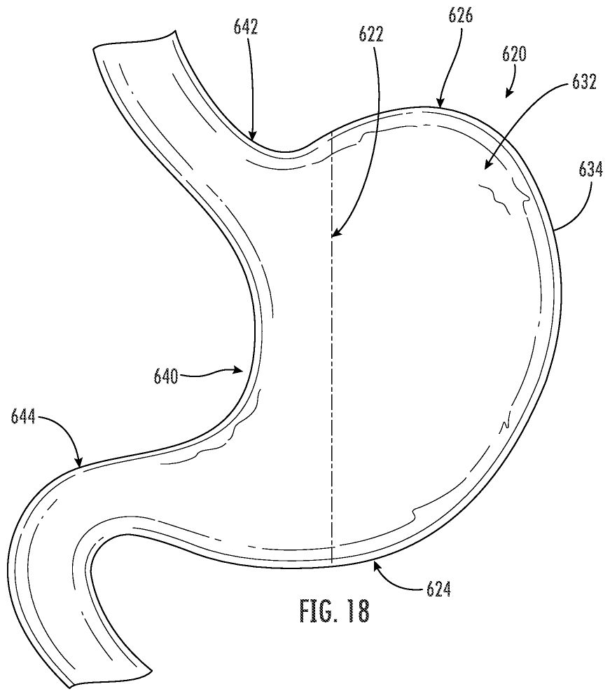

FIGS. 18-20 illustrate a method of using the surgical stapling devices and controls systems herein for a vertical sleeve gastrectomy procedure. FIG. 18 illustrates the anatomy of a stomach 620 and a dissection line 622 for a vertical sleeve gastrectomy. As described from the perspective of the instrument, stomach 620 generally includes a proximal end 624, a distal end 626, a greater curvature 634 and a lesser curvature 640. A fundus 632 and the section of the stomach 620 defined by the greater curvature 634 are the parts of the stomach 620 typically removed during a vertical gastrectomy procedure. The remaining pouch is typically defined by the lesser curvature 640 and dissection line 622. The desired location of dissection line 622 is about 0.5 cm to about 2.0 cm away from a gastroesophageal junction 642 and about 2.0 cm to about 10.0 cm away from a pylorus 644.

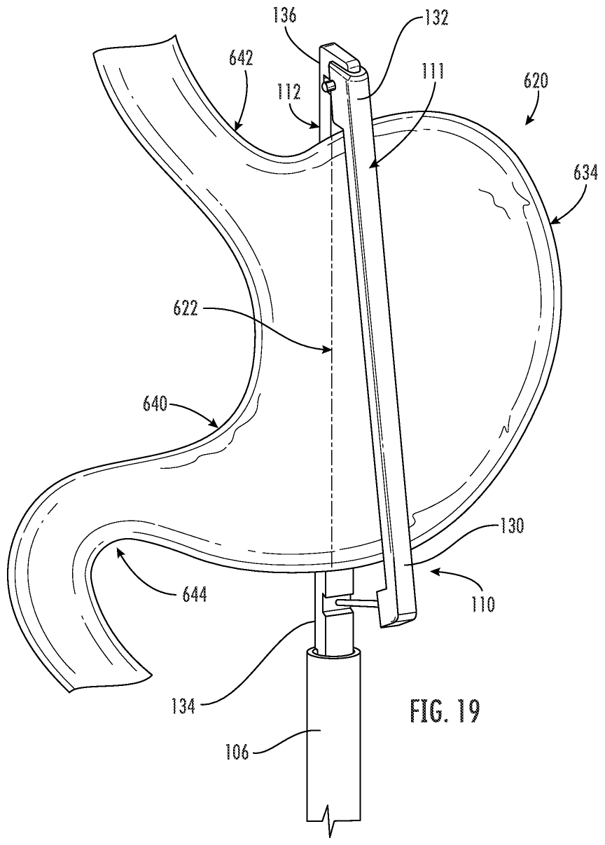

Referring now to FIG. 19, a surgeon first introduces end effector 110 and a portion of shaft 106 of instrument 100 through a suitable portal or entry point (now shown) into the abdominal cavity and positions end effector 110 of surgical instrument 100 around stomach 620 such that jaws 111, 112 are positioned on either side of dissection line 622. As discussed above, end effector 110 may be articulated relative to shaft 106 during this procedure to facilitate the optimal positioning of end effector 110. In particular, end effector 110 may be rotated around longitudinal axis X-X of instrument 100, or it may rotate about one or more axes perpendicular to the longitudinal axis. These articulations may be effected by a proximal actuator that pushes/pulls bands 262 (FIGS. 14A and 14B) such that the bands rotate end effector 110 at wrist 160, or they may be articulated in other suitable means known in the art. In certain embodiments, these articulations may be controlled by control system 600.

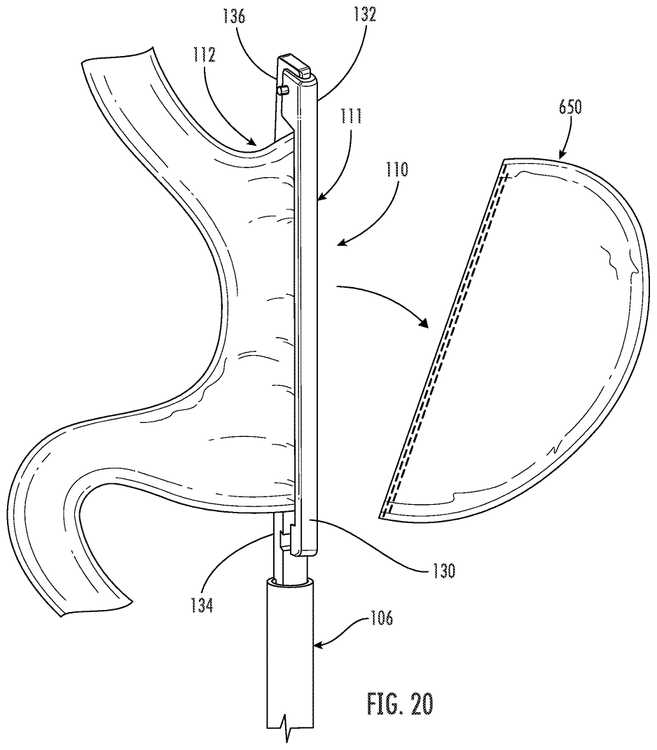

Referring now to FIG. 20, once in position, the surgeon adjusts the gap between distal ends 132, 136 and the proximal ends 130, 134 of jaws 111, 112 until an appropriate gap exists therebetween for dissecting and stapling the stomach tissue. In certain cases, the stomach tissue may vary in thickness along dissection line 622. In these instances, the gap between distal ends 132, 136 may vary from the gap between proximal ends 130, 134.

Control system 600 monitors the torques or forces applied to the proximal and distal ends of jaws 111, 112 as the surgeon adjusts the gaps therebetween. In certain embodiments, control system 600 may include a user interface with one or more indicators that provide an indication that these torques or forces are within the prescribed range for optimal dissection and stapling of the tissue. In one embodiment, for example, the range may be based on a minimum distance representing a threshold force applied by the stomach tissue 620 to the jaws 111, 112 such that the jaws do not compress the tissue too much to effectively apply staples thereto. The ranges may also be based on a maximum distance representing a threshold and/or zero amount of force applied by the tissue to the jaws such that the jaws are not separated so far from each other to be unable to effectively apply staples to the tissue.

Once the ends of jaws 111, 112 are positioned within the prescribed ranges, control system 600 will allow the surgeon to advance drive member 150 to “fire” the device. In one embodiment, third controller 610 inhibits or prevents the advancement of drive member 150 when either of the first or second torques are outside of the first and second ranges, respectively. Drive member 150 may be advanced by actuating rotary input 510 (see FIG. 15), which functions to operate suitable drive mechanisms with backend mechanism 500 that advance drive member 150 through end effector 110. Drive member 150 is advanced distally through end effector 110 to advance shuttle 123 and knife 128 distally through cartridge 122 to staple and cut tissue grasped between jaws 111, 112. In an alternative embodiment, drive member 150 may be advanced proximally from a distal end of end effector 110 to advance shuttle 123 and knife 128 proximally through cartridge 122. As shown in FIG. 20, drive member 150 advances substantially along dissention line 622 to remove a portion 650 of the stomach.

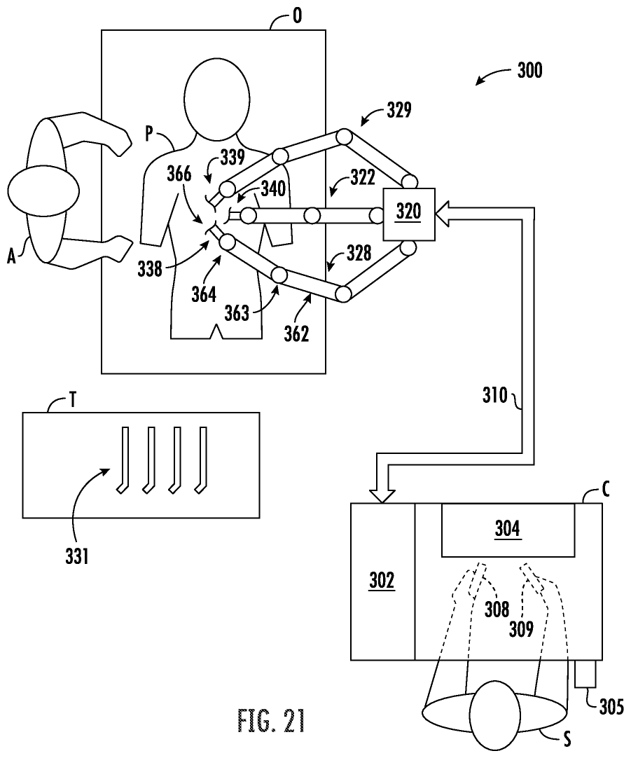

FIG. 21 illustrates, as an example, a top view of an operating room employing a robotic surgical system. The robotic surgical system in this case is a robotic surgical system 300 including a Console (“C”) utilized by a Surgeon (“S”) while performing a minimally invasive diagnostic or surgical procedure, usually with assistance from one or more Assistants (“A”), on a Patient (“P”) who is lying down on an Operating table (“O”).

The servomechanism used for telesurgery will often accept input from two master controllers (one for each of the surgeon's hands) and may include two or more robotic arms. A surgical instrument is mounted on each of the robotic arms. Operative communication between master controllers and associated robotic arm and instrument assemblies is typically achieved through a control system. The control system typically includes at least one processor that relays input commands from the master controllers to the associated robotic arm and instrument assemblies and back in the case of, for example, force feedback or the like. One example of a robotic surgical system is the DA VINCI™ system commercialized by Intuitive Surgical, Inc. of Sunnyvale, California.

A variety of structural arrangements have been used to support the surgical instrument at the surgical site during robotic surgery. The driven coupler or “slave” is often called a robotic surgical manipulator, and exemplary coupler arrangements for use as a robotic surgical manipulator during minimally invasive robotic surgery are described in U.S. Pat. Nos. 7,594,912, 6,758,843, 6,246,200, and 5,800,423, the full disclosures of which are incorporated herein by reference in their entirety for all purposes. These couplers often manipulate an instrument holder to which an instrument having a shaft is mounted. Such a manipulator structure can include a parallelogram coupler portion that generates motion of the instrument holder that is limited to rotation about a pitch axis that intersects a remote center of manipulation located along the length of the instrument shaft. Such a manipulator structure can also include a yaw joint that generates motion of the instrument holder that is limited to rotation about a yaw axis that is perpendicular to the pitch axis and that also intersects the remote center of manipulation. By aligning the remote center of manipulation with the incision point to the internal surgical site (for example, with a trocar or cannula at an abdominal wall during laparoscopic surgery), an end effector of the surgical instrument can be positioned safely by moving the proximal end of the shaft using the manipulator coupler without imposing potentially hazardous forces against the abdominal wall. Alternative manipulator structures are described, for example, in U.S. Pat. Nos. 6,702,805, 6,676,669, 5,855,583, 5,808,665, 5,445,166, and 5,184,601, the full disclosures of which are incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. Manipulation and control of these end effectors is a particularly beneficial aspect of robotic surgical systems. For this reason, it is desirable to provide surgical tools that include mechanisms that provide two or three degrees of rotational movement of an end effector to mimic the natural action of a surgeon's wrist. Such mechanisms should be appropriately sized for use in a minimally invasive procedure and relatively simple in design to reduce possible points of failure. In addition, such mechanisms should provide an adequate range of motion to allow the end effector to be manipulated in a wide variety of positions.

The Console includes a monitor 304 for displaying an image of a surgical site to the Surgeon, left and right manipulatable control devices 308 and 309, a foot pedal 305, and a processor 302. The control devices 308 and 309 may include any one or more of a variety of input devices such as joysticks, gloves, trigger-guns, hand-operated controllers, or the like. The processor 302 may be a dedicated computer that may be integrated into the Console or positioned next to it.

The Surgeon performs a minimally invasive surgical procedure by manipulating the control devices 308 and 309 (also referred to herein as “master manipulators”) so that the processor 302 causes their respectively associated robotic arm assemblies, 328 and 329, (also referred to herein as “slave manipulators”) to manipulate their respective removably coupled surgical instruments 338 and 339 (also referred to herein as “tools”) accordingly, while the Surgeon views the surgical site in 3-D on the Console monitor 304 as it is captured by a stereoscopic endoscope 340.

Each of the tools 338 and 339, as well as the endoscope 340, may be inserted through a cannula or other tool guide (not shown) into the Patient so as to extend down to the surgical site through a corresponding minimally invasive incision such as incision 366. Each of the robotic arms is conventionally formed of links, such as link 362, which are coupled together and manipulated through motor controlled or active joints, such as joint 363.

The number of surgical tools used at one time and consequently, the number of robotic arms being used in the system 300 will generally depend on the diagnostic or surgical procedure and the space constraints within the operating room, among other factors. If it is necessary to change one or more of the tools being used during a procedure, the Assistant may remove the tool no longer being used from its robotic arm, and replace it with another tool 331 from a Tray (“T”) in the operating room.

The monitor 304 may be positioned near the Surgeon's hands so that it will display a projected image that is oriented so that the Surgeon feels that he or she is actually looking directly down onto the operating site. To that end, images of the tools 338 and 339 may appear to be located substantially where the Surgeon's hands are located.

The processor 302 performs various functions in the system 300. One function that it performs is to translate and transfer the mechanical motion of control devices 308 and 309 to their respective robotic arms 328 and 329 through control signals overbus 310 so that the Surgeon can effectively manipulate their respective tools 338 and 339. Another important function is to implement various control system processes as described herein.

Although described as a processor, it is to be appreciated that the processor 302 may be implemented in practice by any combination of hardware, software and firmware. Also, its functions as described herein may be performed by one unit, or divided up among different components, each of which may be implemented in turn by any combination of hardware, software and firmware.

Robotic surgery systems and methods are further described in U.S. Pat. No. 5,797,900, filed on May 16, 1997, issued on Aug. 25, 1998, U.S. Pat. No. 6,132,368, filed on Nov. 21, 1997, issued on Oct. 17, 2000, U.S. Pat. No. 6,331,181, filed on Oct. 15, 1999, issued on Dec. 18, 2001, U.S. Pat. No. 6,441,577, filed on Apr. 3, 2001, issued on Aug. 27, 2002, U.S. Pat. No. 6,902,560, filed on Jan. 6, 2004, issued on Jun. 7, 2005, U.S. Pat. No. 6,936,042, filed on Apr. 16, 2002, issued on Aug. 30, 2005, and U.S. Pat. No. 6,994,703, filed on Dec. 4, 2002, issued on Feb. 7, 2006, the full disclosures of which are incorporated herein by reference for all purposes. A suitable robotic surgical system currently in use is the da Vinci S Surgical System by Intuitive Surgical, Inc.

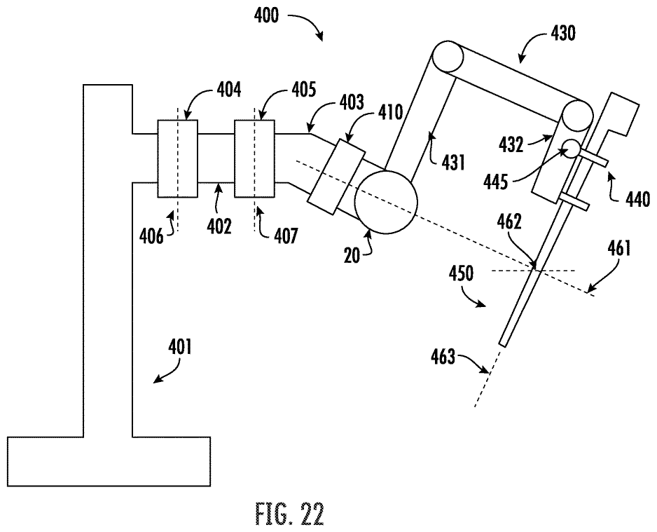

FIG. 22 illustrates, as an example, a side view of a simplified (not necessarily in proportion or complete) illustrative robotic arm assembly 400 (which is representative of robotic arm assemblies 328 and 329) holding a surgical instrument 450 (which is representative of tools 338 and 339) for performing a surgical procedure. The surgical instrument 450 is removably held in tool holder 440. The arm assembly 400 is mechanically supported by a base 401, which may be part of a patient-side movable cart or affixed to the operating table or ceiling. It includes links 402 and 403 which are coupled together and to the base 401 through setup joints 404 and 405.

The setup joints 404 and 405 in this example are passive joints that allow manual positioning of the arm 400 when their brakes are released. For example, setup joint 404 allows link 402 to be manually rotated about axis 406, and setup joint 405 allows link 403 to be manually rotated about axis 407.

Although only two links and two setup joints are shown in this example, more or less of each may be used as appropriate in this and other robotic arm assemblies described herein. For example, although setup joints 404 and 405 are useful for horizontal positioning of the arm 400, additional setup joints may be included and useful for limited vertical and angular positioning of the arm 400. For major vertical positioning of the arm 400, however, the arm 400 may also be slidably moved along the vertical axis of the base 401 and locked in position.

The robotic arm assembly 400 also includes three active joints driven by motors. A yaw joint 410 allows arm section 430 to rotate around an axis 461, and a pitch joint 420 allows arm section 430 to rotate about an axis perpendicular to that of axis 461 and orthogonal to the plane of the drawing. The arm section 430 is configured so that sections 431 and 432 are always parallel to each other as the pitch joint 420 is rotated by its motor. As a consequence, the instrument 450 may be controllably moved by driving the yaw and pitch motors so as to pivot about the pivot point 462, which is generally located through manual positioning of the setup joints 404 and 405 so as to be at the point of incision into the patient. In addition, an insertion gear 445 may be coupled to a linear drive mechanism (not shown) to extend or retract the instrument 450 along its axis 463.

Although each of the yaw, pitch and insertion joints or gears, 410, 420 and 445, is controlled by an individual joint or gear controller, the three controllers are controlled by a common master/slave control system so that the robotic arm assembly 400 (also referred to herein as a “slave manipulator”) may be controlled through user (e.g., surgeon) manipulation of its associated master manipulator.

In some embodiments, the system may include other device control inputs, such as foot pedals and the like, to provide additional control inputs to surgical instrument and/or the endoscope used for the procedure. In some embodiments, the pedals are color designated, usually indicating a cautery action (blue), and cut action (yellow) for bipolar instruments. Sometimes these actions are instrument specific. For example, with a surgical stapler that performs a clamping step (blue), and then a fire step (yellow). The connotation for these colors is generally the same: blue is a lower risk or reversable action, while yellow indicates a high risk, non-reversable action.

For minimally invasive surgery, the instruments must remain substantially stationary with respect to the location at which they enter the patient's body, either at an incision or at a natural orifice, to avoid unnecessary tissue damage. Accordingly, the yaw and pitch motions of each instrument shaft should be centered at a single location on the manipulator assembly roll axis or instrument insertion axis that stays relatively stationary in space. This location is referred to as a remote center of motion. As described in more detail below, a surgical instrument is mounted on and actuated by each surgical instrument manipulator or robotic arm of the manipulator assembly. The instruments are removably mounted so that various instruments may be interchangeably mounted on a particular instrument manipulator. In the representative embodiment, one instrument manipulator is configured to actuate a camera instrument, and three instrument manipulators are configured to actuate various other interchangeable surgical instruments that perform surgical and/or diagnostic work at the surgical site. More or fewer instrument manipulators may be used. In some operational configurations, one or more manipulators may not have an associated surgical instrument during some or all of a surgical procedure.

Each of the joints in the robotic arms and the support structure may include joint brakes that can be manually set (or automatically by the control system) to prevent movement about that particular joint. The base may include a passive, uncontrolled “setup” portion and an actively controlled “manipulator” portion. In one example, the setup portion includes two passive rotational “setup” joints (not shown) which allow manual positioning of the robotic arms when the joint brakes (not shown) are released. A passive prismatic setup joint (not shown) may be used to allow for large vertical adjustments. Alternatively, some of these setup joints may be actively controlled, and more or fewer setup joints may be used in various configurations. The setup joints and links allow a person to place the robotic manipulator portion of the arm at various positions and orientations in Cartesian x, y, z space. The remote center of motion is the location at which yaw, pitch, and roll axes intersect (i.e., the location at which the kinematic chain remains effectively stationary while joints move through their range of motion). As described in more detail below, some of these actively controlled joints are robotic manipulators that are associated with controlling DOFs of individual surgical instruments, and others of these actively controlled joints are associated with controlling DOFs of a single assembly of these robotic manipulators. The active joints and links are movable by motors or other actuators and receive movement control signals that are associated with master arm movements at the surgeon's console. Active joints and manipulator platform move in conjunction and/or independently so that a surgical instrument (or assembly) moves around the remote center of motion at an entry port after the remote center of motion has been established by the passive setup arms and joints. Accordingly, the passive setup joints and links may be used to properly position a remote center of motion with reference to the patient. Once the remote center of motion is properly positioned, brakes at each of the joints are set to prevent the setup portion of the arm from moving.

The system may be configured to generate a general target operating location around the surgical site or OP wherein the instruments will be used within the patient for a particular procedure. The system further generates a central target position or point which is the center of the OP. In one embodiment, the center of the OP is located at the endoscope cannula's remote center of motion. The robotic arms are then restrained from moving beyond a certain distance from the center of the OP to provide the most range of motion (ROM) at the center of the surgical space during the procedure and to limit the risk of instrument collisions. In certain embodiments, the manipulator assembly comprises one or more brakes that prevent the manipulator arms from moving a certain distance away from the OP.

While several embodiments have been shown in the drawings, it is not intended that the disclosure be limited thereto, as it is intended that the disclosure be as broad in scope as the art will allow and that the specification be read likewise. For example, the devices disclosed herein are not limited to the mechanisms described herein for identifying and/or deactivating stapler cartridges. Other suitable devices or mechanisms are described in co-pending and co-owned International Patent Application No. PCT/US19/66513, filed Dec. 16, 2019, and entitled “SURGICAL INSTRUMENTS WITH SWITCHES FOR DEACTIVATING AND/OR IDENTIFYING STAPLER CARTRIDGES”, the complete disclosure of which is herein incorporated by reference in its entirety for all purposes. Therefore, the above description should not be construed as limiting, but merely as exemplifications of presently disclosed embodiments. Thus, the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure.

For example, in a first aspect, a first embodiment is a surgical stapling instrument comprising an elongate shaft having distal and proximal ends and an end effector coupled to the distal end of the shaft and defining a longitudinal axis. The end effector comprises an anvil having a proximal end, a distal end, and a surface positionable on a first side of an anatomical structure, and a cartridge configured to house a plurality of staples and having a proximal end, a distal end and a surface positionable on a second side of the anatomical structure. The instrument further comprises a first coupler movably coupling the proximal end of the anvil to the proximal end of the cartridge and a second coupler movably coupling the distal end of the anvil to the distal end of the cartridge.

A second embodiment is the first embodiment, wherein the first coupler is configured to adjust a distance between the proximal end of the anvil and the proximal end of the cartridge.

A third embodiment is any combination of the first 2 embodiments, wherein the second coupler is configured to adjust a distance between the distal end of the anvil and the distal end of the cartridge.

A 4^{th} embodiment is any combination of the first 3 embodiments, wherein the first coupler is independent from the second coupler.

A 5^{th} embodiment is any combination of the first 4 embodiments, wherein the first coupler comprises a band having a planar first surface and a planar second surface opposite the first surface, the band having a distal end coupled to the proximal end of the anvil and a pulley, wherein a first portion of the band is in contact with the pulley and a second portion of the band is configured to move in a substantially longitudinal direction to adjust the distance between the proximal ends of the anvil and the cartridge.

A 6^{th} embodiment is any combination of the first 5 embodiments, wherein the first coupler pivots the proximal end of the anvil relative to the proximal end of the cartridge from an open position to a substantially closed position.

A 7^{th} embodiment is any combination of the first 6 embodiments, wherein the second coupler comprises a band having a planar first surface and a planar second surface opposite the first surface, the band having a distal end coupled to the distal end of the anvil and a pulley, wherein a first portion of the band is in contact with the pulley and a second portion of the and is configured to move in a substantially longitudinal direction to adjust the distance between the distal ends of the anvil and the cartridge.

An 8^{th} embodiment is any combination of the first 7 embodiments, further comprising a slot in a distal portion of the cartridge and a pin slidable within the slot, wherein the pin is coupled to the anvil to allow the distal end of the anvil to move towards and away from the distal end of the cartridge.

A 9^{th} embodiment is any combination of the first 8 embodiments, wherein a first portion of the band is in contact with the pulley and a second portion of the and is configured to move in a substantially longitudinal direction to adjust the distance between the distal ends of the anvil and the cartridge.

A 10^{th} embodiment is any combination of the first 9 embodiments, further comprising a first spring biasing the distal ends of the anvil and the cartridge away from each other.

An 11^{th} embodiment is any combination of the first 10 embodiments, further comprising a second spring biasing the proximal ends of the anvil and the cartridge away from each other.

A 12^{th} embodiment is any combination of the first 11 embodiments, wherein the first coupler limits a range of distance between the proximal ends of the anvil and the cartridge between a minimum distance and a maximum distance.

A 13^{th} embodiment is any combination of the first 12 embodiments, wherein the second coupler limits a range of distance between the distal ends of the anvil and the cartridge between a minimum distance and a maximum distance.

A 14^{th} embodiment is any combination of the first 13 embodiments, wherein the first coupler comprises an angled slot and a slot pin, wherein the slot pin is coupled to the anvil or the cartridge and configure to advance through the angled slot to adjust a distance between the proximal and distal ends of the anvil and the cartridge.

A 15^{th} embodiment is any combination of the first 5 embodiments, further comprising a close member configured for advancement in a distal direction through the end effector, wherein the slot is disposed on the close member such that the slot pin advanced proximally through the slot as the close member is moved distally.

A 16^{th} embodiment is any combination of the first 15 embodiments, further comprising a drive member configured for advancement in a longitudinal direction through the anvil and the cartridge.

In another aspect, a first embodiment is a surgical stapling instrument comprising an elongate shaft having distal and proximal ends and an end effector coupled to the distal end of the shaft and defining a longitudinal axis. The end effector comprises an anvil having a proximal end, a distal end, and a surface positionable on a first side of an anatomical structure and a cartridge configured to house a plurality of staples and having a proximal end, a distal end and a surface positionable on a second side of the anatomical structure. The instrument further comprises a coupler that movably couples the proximal end of the anvil to the proximal end of the cartridge. The coupler comprises a band having a first planar surface and a second planar surface opposite the first planar surface.

A second embodiment is the first embodiment, wherein the band has a distal end coupled to the anvil and a proximal end extending through the elongate shaft, the coupler further comprising a pulley, wherein a first portion of the band is in contact with the pulley and a second portion of the band is configured to move in a substantially longitudinal direction to adjust the distance between the proximal ends of the anvil and the cartridge.

A 3^{rd} embodiment is any combination of the first two embodiments, wherein the band further comprises a third portion extending between the distal end of the band and the pulley, wherein the third portion extends substantially perpendicular to the longitudinal axis.

A 4^{th} embodiment is any combination of the first 3 embodiments, further comprising a second coupler that movably couples the distal end of the anvil to the distal end of the cartridge, the second coupler comprising a band having a planar first surface and a planar second surface opposite the first surface.

A 5^{th} embodiment is any combination of the first 4 embodiments, wherein the band has a distal end coupled to the anvil and a proximal end extending through the elongate shaft, the second coupler further comprising a pulley, wherein a first portion of the band is in contact with the pulley and a second portion of the band is configured to move in a substantially longitudinal direction to adjust the distance between the distal ends of the anvil and the cartridge.

A 6^{th} embodiment is any combination of the first 5 embodiments, wherein the band further comprises a third portion extending between the distal end of the band and the pulley surface, wherein the third portion extends substantially perpendicular to the longitudinal axis.

In another aspect, a first embodiment is a surgical stapling instrument comprising an elongate shaft having distal and proximal ends and an end effector coupled to the distal end of the shaft and defining a longitudinal axis. The end effector comprises an anvil having a proximal end, a distal end, and a surface positionable on a first side of an anatomical structure and a cartridge configured to house a plurality of staples and having a proximal end, a distal end and a surface positionable on a second side of the anatomical structure. The instrument further comprises a coupler that movably couples the proximal end of the anvil to the proximal end of the cartridge and an articulation element for rotating the end effector relative to the shaft about an axis perpendicular to the longitudinal axis.

A second embodiment is the first embodiment further comprising a second articulation element for rotating the end effector relative to the shaft about a second axis perpendicular to the first axis and perpendicular to the longitudinal axis.

A 3^{rd} embodiment is any combination of the first 2 embodiments, further comprising a third articulation element for rotating the end effector relative to the shaft about the longitudinal axis.

A 4^{th} embodiment is any combination of the first 3 embodiments, wherein the articulation element comprises at least one band having a planar first surface and a planar second surface opposite the first surface.

A 5^{th} embodiment is any combination of the first 4 embodiments, wherein the articulation element comprises a plurality of bands extending longitudinally through the shaft.

A 6^{th} embodiment is any combination of the first 5 embodiments, further comprising a wrist member pivotally coupling the shaft with the end effector, wherein the plurality of bands is twisted relative to each other within the wrist member such that longitudinal movement of the bands within the shaft causes the end effector to rotate relative to the shaft around an axis perpendicular to the longitudinal axis.

A 7^{th} embodiment is any combination of the first 6 embodiments, further comprising a second coupler that movably couples the distal end of the anvil to the distal end of the cartridge, wherein the first engagement element is independent of the second engagement element.

In another aspect, a first embodiment is a system for controlling a surgical stapling instrument having an elongate shaft and an end effector coupled to the distal end of the shaft, the end effector having an anvil and a cartridge having proximal and distal ends movably coupled to each other. The system comprising a first controller configured to detect a first torque applied to the proximal ends of the anvil and the cartridge and to maintain a distance between said proximal ends such that the first torque remains within a first range and a second controller configured to detect a second torque applied to the distal ends of the anvil and the cartridge and to maintain a distance between said distal ends such that the second torque remains within a second range.

A second embodiment is the first embodiment, wherein the stapling instrument further comprises a drive member configured to advance in a longitudinal direction relative to the cartridge and the anvil, the system further comprising a third controller configured to inhibit or prevent advancement of the drive member when either of the first or second torques are outside of the first and second ranges, respectively.

A 3^{rd} embodiment is any combination of the first 2 embodiments, wherein the first and second torques are associated with one or more forces applied to the anvil and the cartridge by tissue disposed therebetween.

A 4^{th} embodiment is any combination of the first 3 embodiments, further comprising a first coupler for adjusting the distance between the proximal ends of the anvil and the cartridge, wherein the first controller is coupled to the first coupler and configured to limit a range of motion of the first coupler based on the first torque relative to the first range.

A 5^{th} embodiment is any combination of the first 4 embodiments, further comprising a second coupler for adjusting the distance between the distal ends of the anvil and the cartridge, wherein the second controller is coupled to the second coupler and configured to limit a range of motion of the second coupler based on the second torque relative to the second range.

A 6^{th} embodiment is any combination of the first 5 embodiments, wherein the first coupler comprises a flexible member having a distal end coupled to the proximal end of the anvil and a proximal end coupled to the first controller, the system further comprising a drive member for withdrawing at least a portion of the flexible member through the shaft to move the proximal end of the anvil towards the proximal end of the cartridge, wherein the first controller is configured to detect the torque applied to the flexible member as the flexible member is withdrawn.

A 7^{th} embodiment is any combination of the first 6 embodiments, wherein the second coupler comprises a flexible member having a distal end coupled to the distal end of the anvil and a proximal end coupled to the second controller, the system further comprising a drive member for withdrawing at least a portion of the flexible member through the shaft to move the distal end of the anvil towards the distal end of the cartridge, wherein the second controller is configured to detect the torque applied to the flexible member as the flexible member is withdrawn.

In another aspect, a first embodiment is a method for controlling a surgical stapling instrument having an elongate shaft and an end effector coupled to the distal end of the shaft, the end effector having an anvil and a cartridge having proximal and distal ends movably coupled to each other. The method comprises detecting a first torque applied to the proximal ends of the anvil and the cartridge, detecting a second torque applied to the distal ends of the anvil and the cartridge and releasing a drive member for advancement through the end effector when the first or second torques are within first and second ranges, respectively.

A second embodiment is the first embodiment further comprising adjusting a distance between the proximal ends of the anvil and the cartridge and detecting the first torque.

A 3^{rd} embodiment is any combination of the first 2 embodiments, further comprising adjusting a distance between the distal ends of the anvil and the cartridge and detecting the second torque.

A 4^{th} embodiment is any combination of the first 3 embodiments, further comprising moving a flexible member relative to the end effector to adjust the distance between the proximal ends of the anvil and the cartridge and preventing further movement of the flexible member when the first torque reaches a threshold level.

A 5^{th} embodiment is any combination of the first 4 embodiments further comprising: moving a flexible member relative to the end effector to adjust the distance between the distal ends of the anvil and the cartridge and preventing further movement of the flexible member when the second torque reaches a threshold level.

A 6^{th} embodiment is any combination of the first 5 embodiments, further comprising preventing the drive member from advancing through the end effector when either of the first or second torques is below a threshold minimum or above a threshold maximum.

In another aspect, a first embodiment is a surgical stapling system comprising an instrument having an elongate shaft and an end effector coupled to the distal end of the shaft, the end effector having an anvil and a cartridge having proximal and distal ends movably coupled to each other and a drive member configured for advancing longitudinally through the end effector. The system comprises a first coupler for adjusting a distance between the distal end of the anvil and the distal end of the cartridge, a second coupler for adjusting a distance between the proximal end of the anvil and the proximal end of the cartridge and an actuator for advancing the drive member in a longitudinal direction relative to the anvil and the cartridge.

A second embodiment is the first embodiment, further comprising a first articulation mechanism for rotating the end effector about a longitudinal axis of the end effector.

A third embodiment is any combination of the first 3 embodiments, further comprising a second articulation mechanism for rotating the end effector relative to the shaft about an axis perpendicular to the longitudinal axis.

A 4^{th} embodiment is any combination of the first 3 embodiments, wherein the second articulation mechanism is configured to rotate the end effector about a yaw axis of the shaft.

A 5^{th} embodiment is any combination of the first 4 embodiments, wherein the second articulation mechanism is configured to rotate the end effector about a pitch axis of the shaft.

A 6^{th} embodiment is any combination of the first 5 embodiments, wherein the first and second couplers each comprise a band having a planar first surface and a planar second surface opposite the first surface and a gear disposed at a proximal end portion of the shaft, wherein the gear is coupled to a rotating disc and configured to translate rotation of the disc into linear movement of the band.

A 7^{th} embodiment is any combination of the first 6 embodiments, further comprising a robotic surgical control system coupled to the rotating discs.

Accordingly, the present disclosure is intended to embrace all such alternatives, modifications, and variances. As well, one skilled in the art will appreciate further features and advantages of the present disclosure based on the above-described embodiments. Accordingly, the present disclosure is not to be limited by what has been particularly shown and described, except as indicated by the appended claims.

1. A surgical stapling instrument comprising:
an elongate shaft having distal and proximal ends;
an end effector coupled to the distal end of the shaft and defining a longitudinal axis, the end effector comprising an anvil having a proximal end, a distal end and a surface positionable on a first side of an anatomical structure and a cartridge configured to house a plurality of staples and having a proximal end, a distal end and a surface positionable on a second side of the anatomical structure;
a first coupler movably coupling the proximal end of the anvil to the proximal end of the cartridge; and
a second coupler movably coupling the distal end of the anvil to the distal end of the cartridge.

2. The stapling instrument of claim 1, wherein the first coupler is configured to adjust a distance between the proximal end of the anvil and the proximal end of the cartridge.

3. The stapling instrument of claim 1, wherein the second coupler is configured to adjust a distance between the distal end of the anvil and the distal end of the cartridge.

4. The stapling instrument of claim 1, wherein the first coupler is independent from the second coupler.

5. The stapling instrument of claim 1, wherein the first coupler comprises:
a band having a planar first surface and a planar second surface opposite the first surface, the band having a distal end coupled to the proximal end of the anvil; and
a pulley, wherein a first portion of the band is in contact with the pulley and a second portion of the band is configured to move in a substantially longitudinal direction to adjust the distance between the proximal ends of the anvil and the cartridge.

6. The stapling instrument of claim 1, wherein the first coupler pivots the proximal end of the anvil relative to the proximal end of the cartridge from an open position to a substantially closed position.

7. The stapling instrument of claim 1, wherein the second coupler comprises:
a band having a planar first surface and a planar second surface opposite the first surface, the band having a distal end coupled to the distal end of the anvil; and
a pulley, wherein a first portion of the band is in contact with the pulley and a second portion of the and is configured to move in a substantially longitudinal direction to adjust the distance between the distal ends of the anvil and the cartridge.

8. The stapling instrument of claim 7, further comprising a slot in a distal portion of the cartridge and a pin slidable within the slot, wherein the pin is coupled to the anvil to allow the distal end of the anvil to move towards and away from the distal end of the cartridge.

9. The stapling instrument of claim 1, further comprising a first spring biasing the distal ends of the anvil and the cartridge away from each other and a second spring biasing the proximal ends of the anvil and the cartridge away from each other.

10. (canceled)

11. The stapling instrument of claim 1, wherein the first coupler limits a range of distance between the proximal ends of the anvil and the cartridge between a minimum distance and a maximum distance and the second coupler limits a range of distance between the distal ends of the anvil and the cartridge between a minimum distance and a maximum distance.

12. (canceled)

13. The stapling instrument of claim 1, wherein the first coupler comprises an angled slot and a slot pin, wherein the slot pin is coupled to the anvil or the cartridge and configure to advance through the angled slot to adjust a distance between the proximal and distal ends of the anvil and the cartridge, the instrument further comprising a close member configured for advancement in a distal direction through the end effector, wherein the slot is disposed on the close member such that the slot pin advanced proximally through the slot as the close member is moved distally.

14. (canceled)

15. (canceled)

16. A surgical stapling instrument comprising:
an elongate shaft having distal and proximal ends; and
an end effector coupled to the distal end of the shaft and defining a longitudinal axis, the end effector comprising:
an anvil having a proximal end, a distal end and a surface positionable on a first side of an anatomical structure;
a cartridge configured to house a plurality of staples and having a proximal end, a distal end and a surface positionable on a second side of the anatomical structure;
a coupler that movably couples the proximal end of the anvil to the proximal end of the cartridge, the coupler comprising a band having a first planar surface and a second planar surface opposite the first planar surface.

17. The stapling instrument of claim 16, wherein the band has a distal end coupled to the anvil and a proximal end extending through the elongate shaft, the coupler further comprising a pulley, wherein a first portion of the band is in contact with the pulley and a second portion of the band is configured to move in a substantially longitudinal direction to adjust the distance between the proximal ends of the anvil and the cartridge.

18. The stapling instrument of claim 17, wherein the band further comprises a third portion extending between the distal end of the band and the pulley, wherein the third portion extends substantially perpendicular to the longitudinal axis.

19. The stapling instrument of claim 16, further comprising a second coupler that movably couples the distal end of the anvil to the distal end of the cartridge, the second coupler comprising a band having a planar first surface and a planar second surface opposite the first surface.

20. The stapling instrument of claim 19, wherein the band has a distal end coupled to the anvil and a proximal end extending through the elongate shaft, the second coupler further comprising a pulley, wherein a first portion of the band is in contact with the pulley and a second portion of the band is configured to move in a substantially longitudinal direction to adjust the distance between the distal ends of the anvil and the cartridge.

21. The stapling instrument of claim 20, wherein the band further comprises a third
portion extending between the distal end of the band and the pulley surface, wherein the third portion extends substantially perpendicular to the longitudinal axis.

22. A surgical stapling instrument comprising:
an elongate shaft having distal and proximal ends; and
an end effector coupled to the distal end of the shaft and defining a longitudinal axis, the end effector comprising:
an anvil having a proximal end, a distal end and a surface positionable on a first side of an anatomical structure;
a cartridge configured to house a plurality of staples and having a proximal end, a distal end and a surface positionable on a second side of the anatomical structure;
a coupler that movably couples the proximal end of the anvil to the proximal end of the cartridge; and
an articulation element for rotating the end effector relative to the shaft about an axis perpendicular to the longitudinal axis.

23. The stapling instrument of claim 22, further comprising a second articulation element for rotating the end effector relative to the shaft about a second axis perpendicular to the first axis and perpendicular to the longitudinal axis and a third articulation element for rotating the end effector relative to the shaft about the longitudinal axis.

24. (canceled)

25. (canceled)

26. The stapling instrument of claim 25, wherein the articulation element comprises a plurality of bands extending longitudinally through the shaft, the instrument further comprising a wrist member pivotally coupling the shaft with the end effector, wherein the plurality of bands is twisted relative to each other within the wrist member such that longitudinal movement of the bands within the shaft causes the end effector to rotate relative to the shaft around an axis perpendicular to the longitudinal axis.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)
